(19) United States
(12) Patent Application Publication
(10) Pub. No.: US 2020/0308540 A1
(43) Pub. Date: Oct. 1, 2020

(54) METHODS OF MAKING, EXPANDING, AND USING A HUMAN PROGENITOR T CELL

(71) Applicants: REGENTS OF THE UNIVERSITY OF MINNESOTA, Minneapolis, MN (US); SUNNYBROOK RESEARCH INSTITUTE, Toronto (CA)

(72) Inventors: Heather Emily Stefanski, Mendota Heights, MN (US); John Edward Wagner, Jr., Wayzata, MN (US); Bruce Robert Blazar, Golden Valley, MN (US); Jastaranpreet Singh, Brampton (CA); Juan Carlos Zúñiga-Pflücker, Toronto (CA)

(21) Appl. No.: 16/651,630

(22) PCT Filed: Sep. 28, 2018
(86) PCT No.: PCT/US2018/053256

§ 371 Date: Mar. 27, 2020

Related U.S. Application Data
(60) Provisional application No. 62/565,257, filed on Sep. 29, 2017.

(51) Int. Cl. C12N 5/0783; A61K 35/17

(52) U.S. Cl. CPC C12N 5/0636; C12N 2506/11; C12N 2501/999; A61K 35/17

(57) ABSTRACT

A CD7⁺ progenitor T cell, a method of producing the CD7⁺ progenitor T cell, and a method of administering the CD7⁺ progenitor T cell. The method of producing the CD7⁺ progenitor T cell includes expanding CD34⁺ cells.

### CONTINUING APPLICATION DATA

This application claims the benefit of U.S. Provisional Application Ser. No. 62/565,257, filed Sep. 29, 2017, which is incorporated by reference herein.

### GOVERNMENT INTEREST

This invention was made with government support under CA065493 and CA142106 awarded by National Institutes of Health. The government has certain rights in the invention.

This invention was made with government support from the Canadian Institutes of Health Research (CIHR).

### BACKGROUND

Bone marrow transplants save lives but do so at a high cost. The chemotherapy and radiation therapies used as part of a pre-transplant regimen often result in impaired immunity, leaving patients susceptible to viral and fungal infections. This susceptibility causes significant morbidity and mortality. An effective immune response against these infections requires functional T lymphocytes (also referred to as T cells). T lymphocytes are critical not only for fighting infection but also for preventing relapse. Many investigators have examined the effects of increasing the number of stem cells in transplant patients to expedite neutrophil recovery. In contrast, increasing donor T cell number has proved to be more difficult because of the increased risk of graft versus host disease. Currently there is a clinical gap in therapeutic treatment options to increase T cell numbers safely and effectively post-transplant.

### SUMMARY OF THE INVENTION

This disclosure describes a progenitor T cell, a method of producing the progenitor T cell, and a method of administering the progenitor T cell. In some embodiments, the progenitor T cell may be administered to a subject having a condition requiring an increase in the number of T cells including, for example, a subject who has undergone chemotherapy or radiation therapy and/or a patient undergoing a bone marrow transplant.

In one aspect, this disclosure describes a method that includes culturing stem cells or progenitor cells with a compound that promotes expansion of CD34⁺ cells to produce an expanded population of cells; and culturing the expanded population of cells with a cell that expresses a Notch ligand to produce a CD7⁺ progenitor T cell. In some embodiments, the stem cells include hematopoietic stem cells.

In some embodiments, the compound that promotes expansion of CD34⁺ cells includes an aryl hydrocarbon receptor antagonist and/or a pyrimidoindole derivative. In some embodiments, the compound that promotes expansion of CD34⁺ cells includes one or more of SR1, an SR1-derivative, UM171, and UM729.

In some embodiments, the CD7⁺ progenitor T cell expresses at least one of CD1a and CD5. In some embodiments, the CD7⁺ progenitor T cell does not express CD34. In some embodiments, the CD7⁺ progenitor T cell expresses a diminished level of CD34 expression compared to a non-expanded population of cells. In some embodiments, the expanded population of cells includes at least 90 percent (%) CD34⁻ cells or at least 95% CD34⁻ cells.

In some embodiments, a cell that expresses a Notch ligand includes an OP9 cell. In some embodiments, the Notch ligand includes at least one of DL1 or DL4. In some embodiments, a cell that expresses a Notch ligand includes an OP9-DL1 cell or an OP9-DL4 cell or both.

In some embodiments, the method includes isolating the stem cells or progenitor cells from one or more of umbilical cord blood, peripheral blood, an induced pluripotent stem cell (iPSC), an embryonic stem cell, and bone marrow. In some embodiments, the method does not include selection of CD34⁺ cells. In some embodiments, the stem cells include hematopoietic stem cells.

In another aspect, this disclosure describes a method that includes administering the CD7⁺ progenitor T cell to a mammal. In some embodiments, the method includes administering umbilical cord blood cells, CD34⁺ cells enriched from umbilical cord blood, and/or hematopoietic stem cells to the mammal in addition to the CD7⁺ progenitor T cell. In some embodiments, the method includes expanding the HSCs with an aryl hydrocarbon receptor antagonist prior to administering the HSCs to the mammal. In some embodiments, wherein the stem cells or progenitor cells are derived from umbilical cord blood, the umbilical cord blood cells, CD34⁺ cells enriched from umbilical cord blood, and/or the HSCs may be derived from the same umbilical cord.

In a further aspect, this disclosure describes a CD7⁺ progenitor T cell produced by the methods disclosed herein and a composition including the CD7⁺ progenitor T cell. In some embodiments a composition including the CD7⁺ progenitor T cell may further include umbilical cord blood (UCB) cells. In some embodiments, the composition including the CD7⁺ progenitor T cell may be administered to a mammal.

In an additional aspect, this disclosure describes an isolated CD34⁻CD7⁺ progenitor T cell. In some embodiments, the isolated CD34⁻CD7⁺ progenitor T cell is capable of engraftment into a thymus. In some embodiments, the isolated CD34⁻CD7⁺ progenitor T cell includes an aryl hydrocarbon receptor antagonist-expanded CD34⁻CD7⁺ progenitor T cell including, for example, an SR1-expanded CD34⁻CD7⁺ progenitor T cell.

A “progenitor T cell” (also referred to herein as “Tprogenitor,” “T-progenitor,” “ProT cell,” or “proT-cell”) is a cell capable of maturing in to a mature T cell. In some embodiments, the progenitor T cell is preferably CD7⁺. In some embodiments, the progenitor T cell is CD44⁺, CD117⁺, CD135⁺, Sca-1⁺, CD24⁺, CD27+, CD45R⁺, CD5, CD1a, and/or CD62L⁺.

In some embodiments, a “diminished level” or a “diminished level of expression” can refer to expression that is reduced by at least 5 percent (%), at least 10%, at least 25%, at least 50%, at least 75%, at least 80%, at least 90%, at least 95%, at least 97%, or at least 99%.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows a schematic of an exemplary approach to produce and evaluate progenitor T cells (also referred to herein as “Tprogenitor,” “T-progenitor,” “ProT cell,” or “proT-cell”). CD34⁺ cells are isolated from a stem cell source (e.g., umbilical cord blood, peripheral blood, an induced pluripotent stem cell (iPSC), an embryonic stem cell, bone marrow, etc.). The CD34⁺ cells are placed in culture and expanded (e.g., with a drug, protein, small molecule, or RNA). In an exemplary embodiment, the cells are expanded for 15 days. The expanded cells are then cultured on cells that express a Notch ligand (e.g., OP9-DL1 cells or OP9-DL4 cells). In an exemplary embodiment, the cells are cultured on cells that express a Notch ligand for 14 to 21 days. The expanded cells may be cultured on cells that express a Notch ligand in the presence of FLT-3 ligand (FLT-3L) (e.g., 5 ng/ml), IL-7 (e.g., 5 ng/ml), and/or human SCF (e.g., 50 ng/ml). The resulting progenitor T cells are then harvested and injected at different concentrations (e.g., 2×10⁵ to 10×10⁶) into an immunodeficient animal (e.g., an irradiated immunodeficient mouse). In some embodiments, the cells may be injected along with CD34⁺ hematopoietic stem cells (HSCs) (e.g., 2×10⁴). The mice can be sacrificed (e.g., 4-12 weeks later), and thymus and spleen evaluated for percentage of human CD45⁺ cells.

FIG. 2 shows SR1 expansion of umbilical cord blood (UCB) results in increased numbers of progenitor T cells after culture on OP9-DL1 cells compared to the number of progenitor T cells generated without SR1 expansion. Briefly CD34⁺ cells were placed on OP9-DL1 cells without pretreatment (naïve UCB) or CD34⁺ cells were expanded in the presence of SR1 and then placed on OP9-DL1 cells (SR1). The resulting number of T progenitor cells was measured, and greater than 90% of the culture was determined to be a proT cell (CD7⁺ cell), as measured by flow cytometry.

FIG. 3 shows SR1-expanded cells lose CD34 expression in vitro during OP9-DL1 culture. CD34⁺ cells were placed on OP9-DL1 cells without pretreatment (naïve UCB) or CD34⁺ cells were expanded in the presence of SR1 and then placed on OP9-DL1 cells (SR1). Cells were harvested at certain time points during the co-culture and assessed for their expression of CD34 and CD7. At each time point tested, SR1-treated cells demonstrated a different phenotype with respect to CD34 and CD7 expression than cells that were not pretreated with SR1.

FIG. 4 shows that progenitor T cells derived from both untreated and SR1-expanded cord blood demonstrate T cell thymic engraftment. Briefly CD34⁺ cells were placed on OP9-DL1 cells without pretreatment (naïve UCB) or CD34⁺ cells were expanded in the presence of SR1 and then placed on OP9-DL1 cells (SR1). After 21 days of co-culture with OP9-DL1 cells, the resulting T-progenitors were injected into irradiated (120 cGy) immunodeficient (NOD/SCID/γcnull (NSG)) mice (3 mice per group) at the indicated concentrations along with 2×10⁴ CD34⁺ hematopoietic stem cells (HSCs). 12 weeks later, the mice were sacrificed, and the ability of the cells to engraft the thymus was assessed by measuring CD4 and CD8 expression in CD45⁺ cells in the thymus.

FIG. 5 shows progenitor T cells derived from both untreated and SR1-expanded cord blood demonstrate peripheral T cell engraftment in the spleen. Briefly CD34⁺ cells were placed on OP9-DL1 cells without pretreatment (naïve UCB) or CD34⁺ cells were expanded in the presence of SR1 and then placed on OP9-DL1 cells (SR1). After 21 days of co-culture with OP9-DL1 cells, the resulting T-progenitors were injected into irradiated (120 cGy) NSG mice (3 mice per group) at the indicated concentrations along with 2×10⁴ CD34⁺ hematopoietic stem cells (HSCs). 12 weeks later, the mice were sacrificed, and the ability of the cells to engraft the spleen was assessed by measuring CD4 and CD8 expression in CD45⁺ cells in splenocytes.

FIG. 6 shows progenitor T cells from SR1-expanded cord blood demonstrate both thymic and peripheral T cell engraftment. Briefly CD34⁺ cells were placed on OP9-DL1 cells without pretreatment (naïve UCB, unfilled columns) or CD34⁺ cells were expanded in the presence of SR1 and then placed on OP9-DL1 cells (SR1, filled columns). After 21 days of co-culture with OP9-DL1 cells, the progenitor T cells were injected into irradiated (120 cGy) NSG mice at the indicated concentrations along with 2×10⁴ CD34⁺ hematopoietic stem cells (HSCs). 12 weeks later, the mice were sacrificed, and the ability of the cells to engraft the thymus and spleen was assessed by flow cytometry. The only significant difference in engraftment between naïve and SR1 cells was observed in the thymus of mice that received 5×10⁶ cells.

FIG. 7 shows that UM171-expanded cells, like SR1-expanded cells, lose CD34 expression during culture with OP9-DL1 cells. CD34⁺ cells were expanded in the presence of SR1 or UM171 and then placed on OP9-DL1 cells. Cells were harvested after 21 days in culture, and CD34 and CD7 expression was assessed. Cells that were expanded by SR1 and UM171 demonstrate a similar phenotype.

FIG. 8 shows a schematic of an exemplary approach to produce, sort, and evaluate progenitor T cells, as further described in Example 6.

FIG. 9 shows sorted CD34⁻CD7⁺ Tprogenitors from SR1-expanded cord blood can engraft in the thymus. The top panels show the percentage of live human CD45⁺ cells from one representative mouse that received CD34⁺CD7⁺ cells and two mice that received CD34⁻CD7⁺ cells. The bottom panels show exemplary CD8 vs CD4 dot plots from one representative mouse that received CD34⁺CD7⁺ cells and two mice that received CD34⁻CD7⁺ cells, as described in Example 6. Thymic cellularity is indicated in the bar graph; * indicates p<0.05; n=at least 4 mice per group

FIG. 10 shows CD34⁻⁺ Tprogenitors from naïve UCB do not result in thymic engraftment. The top panels show the percentage of live human CD45⁺ cells from one representative mouse that received CD34⁺CD7⁺ cells and two mice that received CD34⁻CD7⁺ cells. The bottom panels show exemplary CD8 vs CD4 dot plots showing no thymic engraftment in mice that received CD34-CD7+ cells. Shown is one representative mouse that received CD34⁻CD7⁺ cells and two mice that received CD34⁻CD7⁺ cells, as described in Comparative Example 1.

FIG. 11A-FIG. 11H show SR1-expanded HSPCs can develop into T-lineage progenitors in vitro and engraft in vivo despite reduced CD34⁺CD7⁺ co-expression compared to nave-HSPCs. FIG. 11A. An exemplary outline for in vitro SR1-HSPC expansion (15 days) followed by progenitor T-cell expansion for 14 days on irradiated OP9-DL1 cells, as further described in Example 7. FIG. 11B. Exemplary flow cytometric analysis for the expression of CD34, CD7, CD5 and CD1a from co-cultures for early T-progenitor expansion. FIG. 11C. Proportion of CD34⁺CD7⁺, CD34⁻CD7⁺, CD7³⁰ CD5⁺ and CD7⁺CD1a⁺ subsets in Naïve-UCB co-cultures compared to SR1-UCB co-cultures. FIG. 11D. Fold cell expansion of naïve versus SR1-expanded HSPCs in OP9-DL1 co-culture. FIG. 11E. Fold cell expansion of naïve versus SR1-expanded HSPCs in OP9-DL1 co-culture normalized to initial CD34⁺ input cell number. The results shown are representative of at least 3 independent experiments. Asterisks represent statistical significance as determined by t-tests (*p<0.05; **p<0.005). FIG. 11F. SR1-expanded HSPCs or naïve HSPCs were differentiated on OP9-DL1 cells for 14 days, and CD34⁺CD7⁺ and CD34⁻CD7⁺ cells were sorted by flow cytometry. Neonatal NSG mice were injected intra-hepatically with 1.0×10⁶ cells of either subset. FIG. 11G. Thymuses were harvested after 4 weeks and cells were stained for CD45,CD4 and CD8. Flow cytometric analysis of live (DAPI⁻CD45⁺) cells for the expression of CD4 and CD8 are shown from mice transplanted with either subset as indicated. FIG. 11H. Thymus cellularity for transplanted mice. The results shown are representative of at least 2 independent experiments. Asterisks represent statistical significance as determined by two-way ANOVA (*p<0.05).

FIG. 12A-FIG. 12G shows SR1-CD7⁺ cells home to the thymus and mature in vivo and have equal homing capabilities compared to naïve-CD7⁺ cells. As further described in Example 7, thymuses were harvested from mice (n=4) at 4 weeks (FIG. 12A) or 10-12 weeks (FIG. 12B) post-injection of SR1-CD7⁺ cells, and the percentage of live CD45+ cells and CD4 vs CD8 are shown. FIG. 12C. Representative flow cytometry plots of CD3 expression on circulating human CD45⁺ cells harvested from the spleen of mice (n=3) 10-12 weeks after injection of CD7⁺ cells. FIG. 12D. Representative flow cytometry plots for intracellular IL-2, IFN-γ, and TNF-α upon in vitro stimulation (6 hours) of human CD45⁺ CD3⁺ cells harvested from the spleen after 10-12 weeks. The results shown are representative of at least 3 independent experiments. FIG. 12E. A 1:1 mixture of sorted ZsGreen⁺ naïve-CD7⁺-cells (3.0×10⁵) and sorted ZsGreen⁻(3.0×10⁵) SR1-CD7⁺-cells were injected into non-irradiated NSG neonatal mice and the thymuses harvested and analyzed after 4 weeks (n=3 mice for naïve alone or naïve+SR1, n=4 mice for SR1 alone). FIG. 12F. Flow cytometric analysis of human CD45 and ZsGreen expression, and CD4 vs CD8 expression on CD45⁺ZsGreen⁺- and CD45⁺ZsGreen⁻-gated cells for naïve-CD7⁺ or SR1-CD7⁺-derived cells, respectively. FIG. 12G. Percentage of ZsGreen⁻ or ZsGreen⁺ cells as a proportion of total human CD45⁺cells for individual mice shown. The results shown are representative of at least 3 independent experiments.

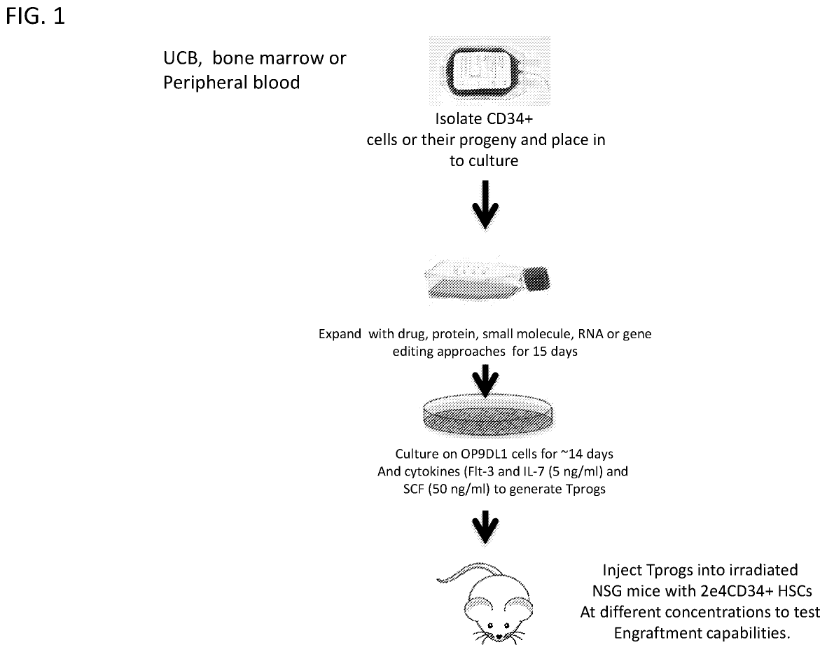

### DETAILED DESCRIPTION

This disclosure describes a progenitor T cell (also referred to herein as “Tprogenitor,” “T-progenitor,” “ProT cell,” or “proT-cell”), a method of producing the progenitor T cell, and a method of administering the progenitor T cell. In some embodiments, the progenitor T cell is preferably CD7⁺. Although progenitor T cells have previously been differentiated from a human umbilical cord blood (UCB)-derived hematopoietic stem cells using coculture with OP9-DL1 cells, such coculture often produces an inadequate number of progenitor T cells for therapeutic uses.

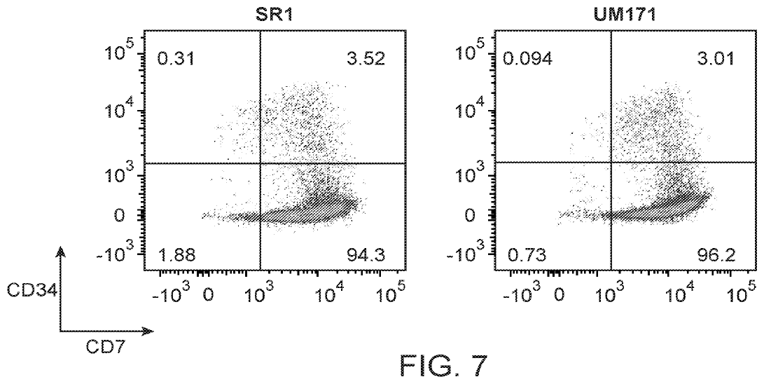

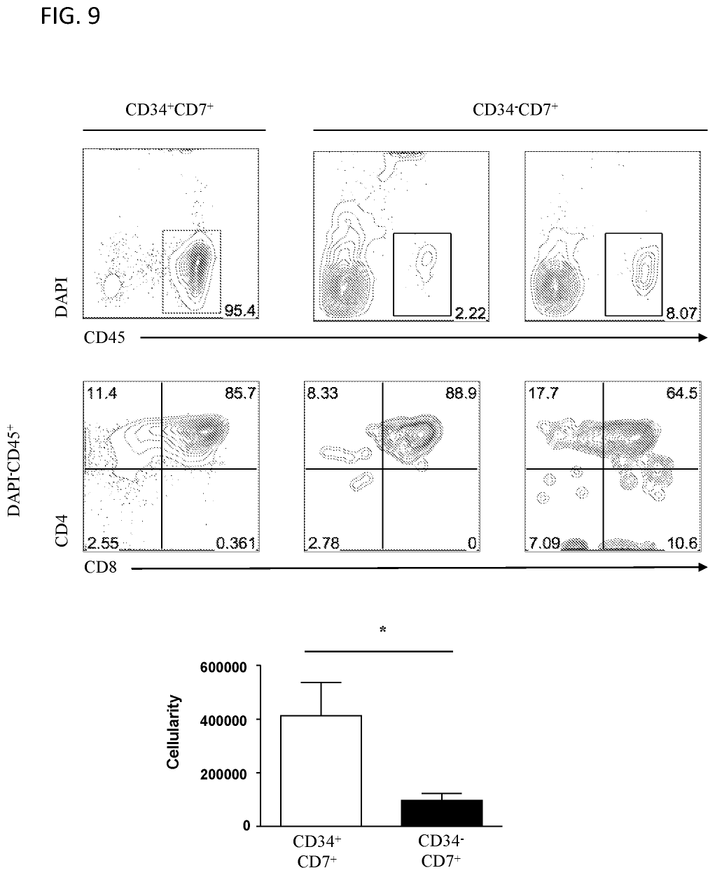

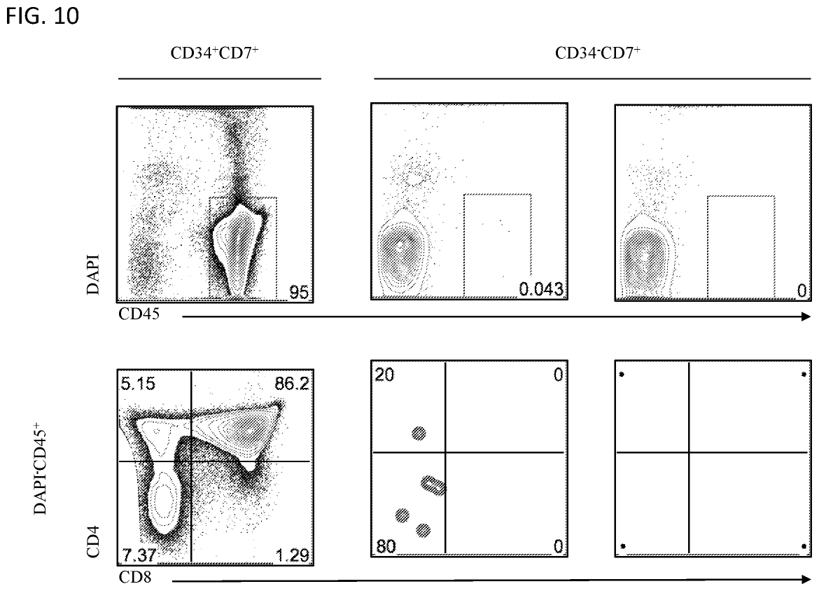

This disclosure describes using a compound that promotes expansion of CD34⁺ cells (including, for example, the aryl hydrocarbon antagonist Stem Reginin 1 (SR1) and/or UM171) to produce an expanded population of cells before culturing the expanded population of cells with an OP9-DL1 cell or another cell that expresses a Notch ligand. Surprisingly, and as shown, for example, in FIG. 7, although the expanded population of cells exhibits a very low frequency (e.g., <3%) of CD34⁺CD7⁺ cells after co-culture with OP9-DL1—a frequency of CD34⁺ cells previously thought to be too low to be engraftable and/or therapeutically useful—cells produced by the methods disclosed herein demonstrate both thymic and peripheral T cell engraftment. Also unexpectedly, and as shown in, for example, FIG. 9, CD34⁻CD7⁺ cells from the expanded population of cells—a population of cells previously thought not to be engraftable and/or therapeutically useful—do engraft in the thymus. These results are in marked contrast to CD34⁻CD7⁺ cells from a non-expanded population of cell which, as shown in FIG. 10, do not engraft in the thymus.

Despite advances in drug discovery, an intact immune system is required for functional immunity post bone marrow transplant. Progenitor T cells have the potential to decrease the risk of relapse of leukemia or other types of cancer in bone marrow transplant patients and to decrease the number of infections post-transplant that cause significant morbidity and mortality in patients. For example, progenitor T cell adoptive transfer with hematopoietic stem/progenitor cells (HSPCs) enhanced HSPC-derived T-cell reconstitution in a pre-clinical hematopoietic stem cell transplantation model (Awong et al. Blood. 2013; 122(26):4210-4219; Zakrzewski et al. Nat Med. 2006; 12(9):1039-1047), suggesting that progenitor T cell adoptive transfer may overcome post-hematopoietic stem cell transplantation immunodeficiency (Awong et al. Curr Opin Hematol. 2010; 17(4):327-332) if sufficient progenitor T cells can be generated in vitro from a single umbilical cord blood unit.

Notch1-based culture systems have been used to generate committed progenitor T cells in vitro. (See, e.g., U.S. Pat. No. 8,772,028, which is incorporated herein by reference.) For example, the OP9-DL1 co-culture system uses a bone marrow stromal cell line (OP9) transduced with the Notch ligand delta-like-1 (DL-1) to support T cell development from multiple stem cell sources including human umbilical cord blood (UCB). Initially, ex vivo expansion of ProT cells using the co-culture system and adoptive transfer of mouse ProT cells was found to enhance immune reconstitution after bone marrow transplant (BMT). (Zakrzewski et al., Nat. Med. 2006;12(9):1039-47; Zakrzewski et al., Nat. Biotechnol. 2008; 26(4):453-61.) This work has since been translated to humans. (Awong et al., Blood 2009;114(5):972-82; Awong et al. Blood 2013;122(26):4210-9.) Although using progenitor T cells derived from umbilical cord blood (UCB) showed potential for providing an adoptive therapy to enhance the poor immune system of transplant patients, the small, finite number of stem cells available from umbilical cord blood limits the number of progenitor T cells that may be generated and the numbers generated are insufficient for clinical trials. This disclosure describes a method of producing a progenitor T cell that includes expanding the cells prior to co-culture with a cell expressing a Notch ligand. Surprisingly, despite the loss of CD34 expression, which was believed to be required for successful engraftment—the progenitor T cells generated using the methods described herein are capable of successful engraftment and are generated in much greater numbers than progenitor T cells derived using the other methods available at the time of the invention.

In one aspect, this disclosure describes a method that includes producing a progenitor T cell. In some embodiments, the progenitor T cell is preferably CD7⁺. In some embodiments, the progenitor T cell does not express CD34 or expresses a diminished level of CD34. In some embodiments, the progenitor T cell expresses CD1a and/or CD5.

The method includes culturing stem cells and/or progenitor cells with a compound that promotes expansion of CD34⁺ cells to produce an “expanded population of cells.” The method further includes culturing the expanded population of cells with a cell that expresses a Notch ligand.

The stem cells or progenitor cells may be derived from any suitable source that includes CD34⁺ cells. In some embodiments, the method includes isolating the stem cells or progenitor cells from one or more of umbilical cord blood, peripheral blood, an induced pluripotent stem cell (iPSC), an embryonic stem cell, and bone marrow.

In some embodiments, the stem cells or progenitor cells may be derived from umbilical cord blood (UCB), peripheral blood, an induced pluripotent stem cell (iPSC), an embryonic stem cell, and/or bone marrow. In some embodiments, the stem cells or progenitor cells are preferably derived from human umbilical cord blood. In some embodiments, the stem cells preferably include hematopoietic stem cells (HSCs). In some embodiments, the stem cells or progenitor cells preferably include CD34⁺ cells. In some embodiments, the stem cells or progenitor cells preferably include a population of cells from UCB, peripheral blood, an induced pluripotent stem cell (iPSC), an embryonic stem cell, and/or bone marrow enriched for CD34⁺ cells.

The expanded population of cells is created by exposing the stem cells or progenitor cells to a compound that promotes expansion of CD34⁺cells. In some embodiments, the compound includes an aryl hydrocarbon receptor antagonist including, for example, SR1 or an SR1-derivative. Surprising, SR1 expansion of human umbilical cord blood prior to co-culture with a cell that expresses a Notch ligand results in a 2000-fold increase in ProT cells during co-culture—without the addition of SR1 to that co-culture. This expansion results in billions of ProT cells.

SR1 has previously been shown to result in a 330-median fold expansion of CD34+ stem cells. (Wagner et al., Cell Stem Cell. 2016;18(1):144-55.) In a Phase I/II trial using SR1-expanded cord blood, SR1 produced a 330-fold increase in CD34⁺ cells and led to engraftment in 17of 17 patients at a median of 15 days for neutrophils and 49 days for platelets, significantly faster than in patients treated with unmanipulated UCB. In contrast to the previous expansion observed for of CD34⁺ stem cells treated with SR1, SR1 expansion of human umbilical cord blood followed by co-culture with a cell that expresses a Notch ligand unexpectedly results in continued expansion of cells during co-culture—without the addition of SR1 to that co-culture—and results in a 2000-fold increase in ProT cells.

The compound that promotes expansion of CD34⁺ cells may include, for example, a drug, a protein, a small molecule, or an RNA. In some embodiments, the compound that promotes expansion of CD34⁺ cells includes an aryl hydrocarbon receptor antagonist. In some embodiments, the compound that promotes expansion of CD34⁺ cells includes SR1 or a derivative of SR1 or both. In some embodiments, the compound that promotes expansion of CD34⁺ cells includes a pyrimidoindole derivative including, for example, UM171 or UM729. As shown, for example, in FIG. 7, expansion with either SR1 or UM171, compounds that promote expansion of CD34⁺ cells, results in similar phenotypes during culture with OP9-DL1 cells. After co-culture with OP9-DL1 cells, cells treated with either SR1 or UM171 are also able to engraft the thymus.

In some embodiments, the expanded population of cells exhibit a diminished level of CD34 expression, minimal CD34 expression, or no CD34 expression. In some embodiments, the expanded population of cells exhibit a diminished level of CD34 expression, minimal CD34 expression, or no CD34 expression compared to a non-expanded population of cells where the “non-expanded population of cells” includes the same starting stem cells or progenitor cells that have not been incubated with or exposed to a compound that promotes expansion of CD34⁺ cells. In some embodiments, CD34 expression is diminished by at least 50%, at least 60%, at least 70%, at least 75%, at least 80%, or at least 90% compared to the CD34 expression of a non-expanded population of cells. In some embodiments, the expanded population of cells includes at least 80% cells, at least 90% cells, at least 95% cells, at least 97% cells, at least 98% cells, or at least 99% cells that are CD34⁻ cells. In some embodiments, the expanded population of cells the expanded population of cells includes at least 80% cells, at least 90% cells, at least 95% cells, at least 97% cells, at least 98% cells, or at least 99% cells that are CD34⁺ cells. In some embodiments, CD34⁻ cells may be selected and/or sorted from the expanded population of cells.

In some embodiments, the expanded population of cells exhibit a diminished level of CD34 expression, minimal CD34 expression, or no CD34 expression compared to a non-expanded population of cells that has been cultured with a cell that expresses a Notch ligand progenitor T cell. In some embodiments, CD34 expression is diminished by at least 50%, at least 60%, at least 70%, at least 75%, at least 80%, or at least 90% compared to the CD34 expression of a non-expanded population of cells. In some embodiments, the expanded population of cells includes at least 80% cells, at least 90% cells, at least 95% cells, at least 97% cells, at least 98% cells, or at least 99% cells that are CD34⁻ cells. In some embodiments, the expanded population of cells the expanded population of cells includes at least 80% cells, at least 90% cells, at least 95% cells, at least 97% cells, at least 98% cells, or at least 99% cells that are CD34⁻ CD7⁺ cells. In some embodiments, CD34⁻ cells may be selected and/or sorted from the expanded population of cells that has been cultured with a cell that expresses a Notch ligand progenitor T cell.

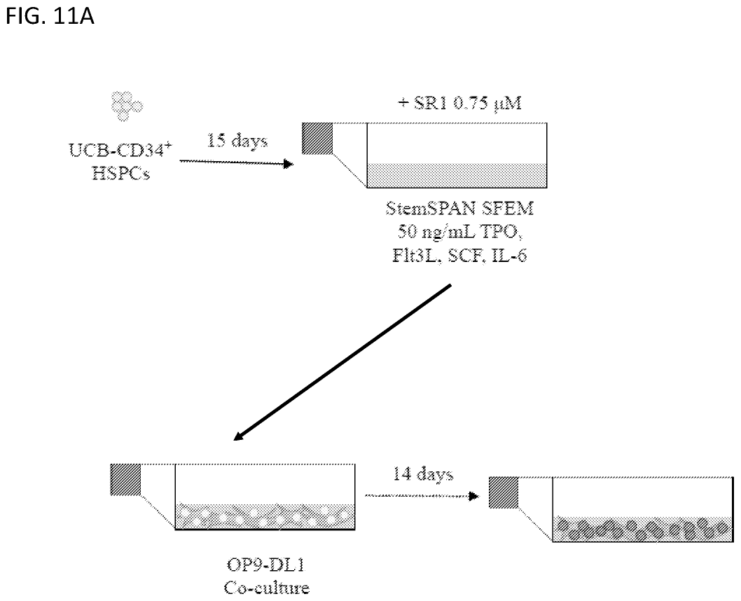

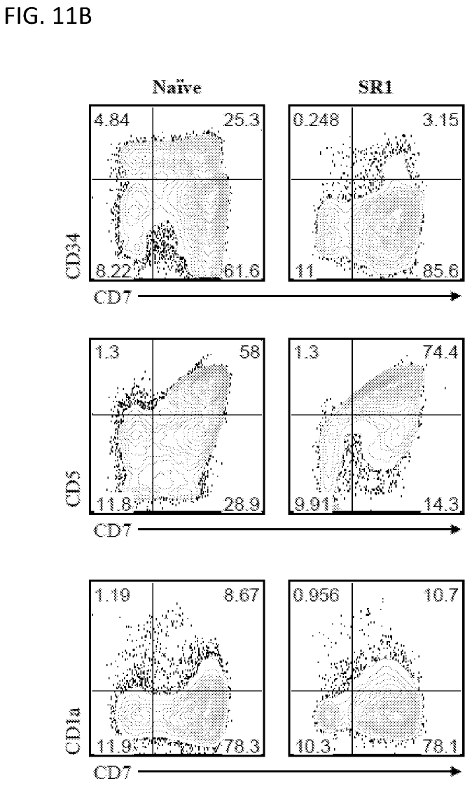

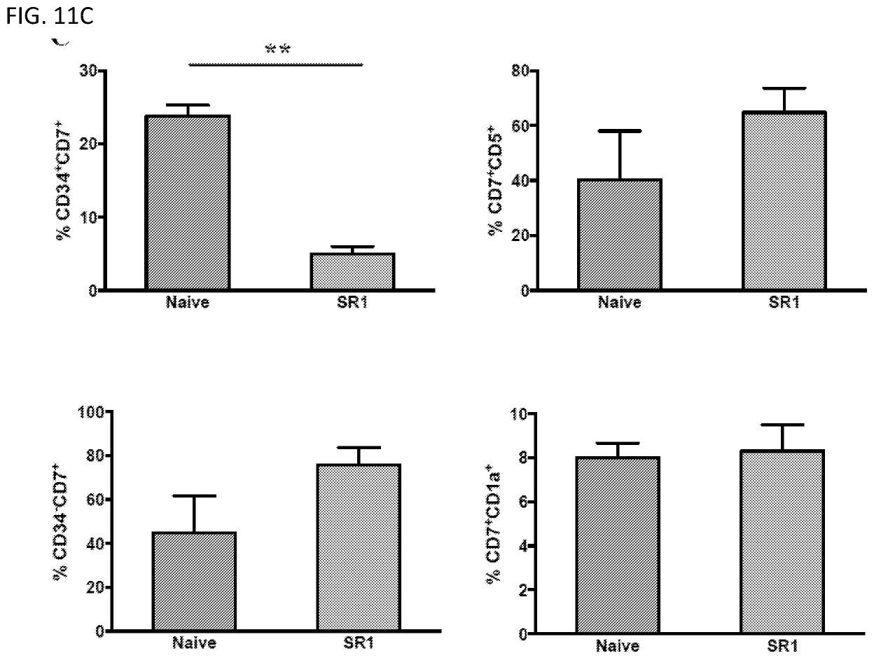

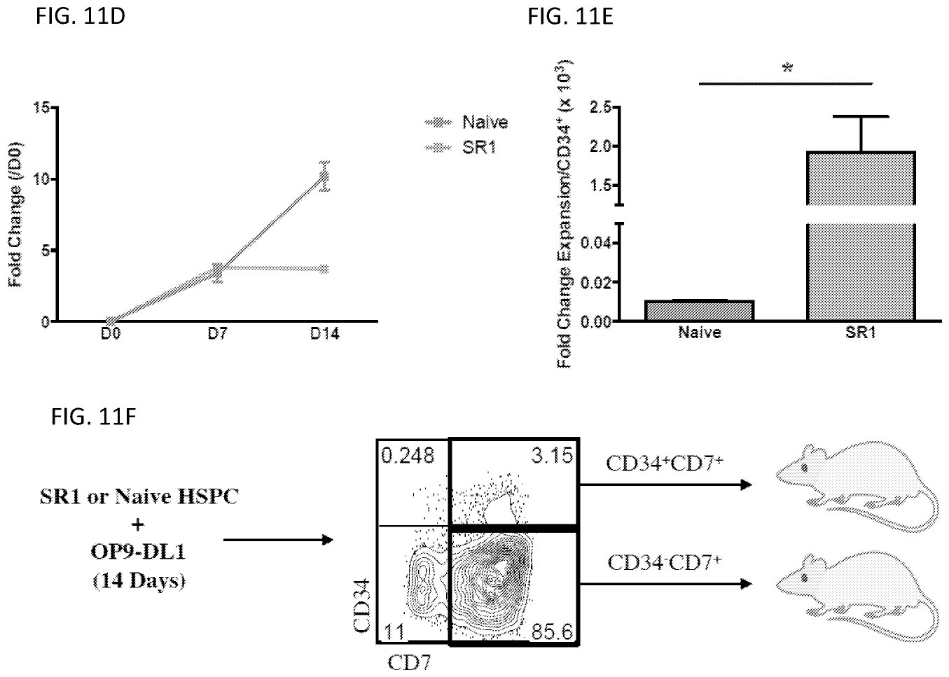

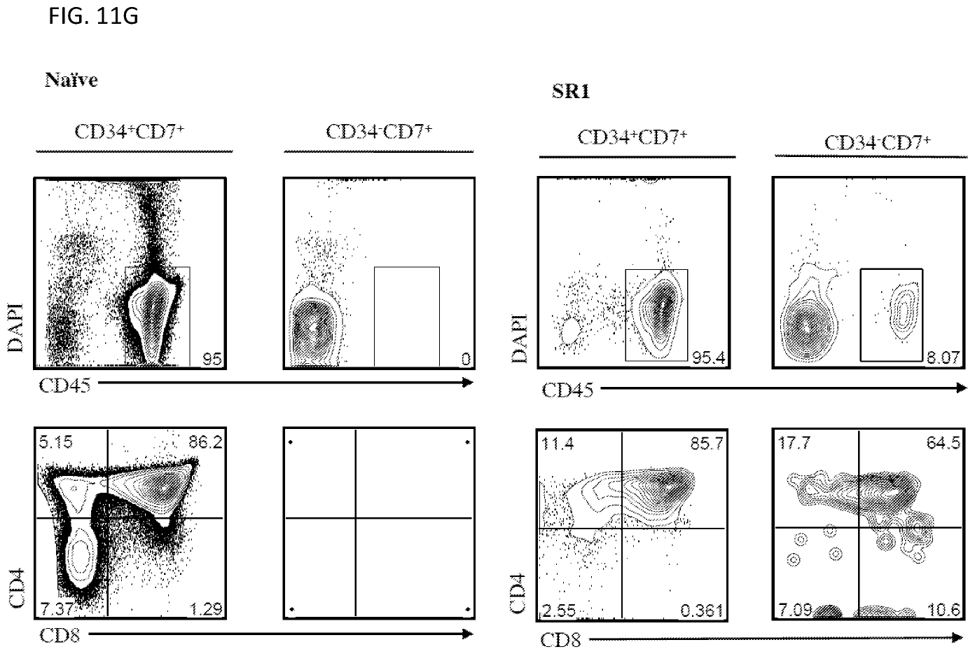

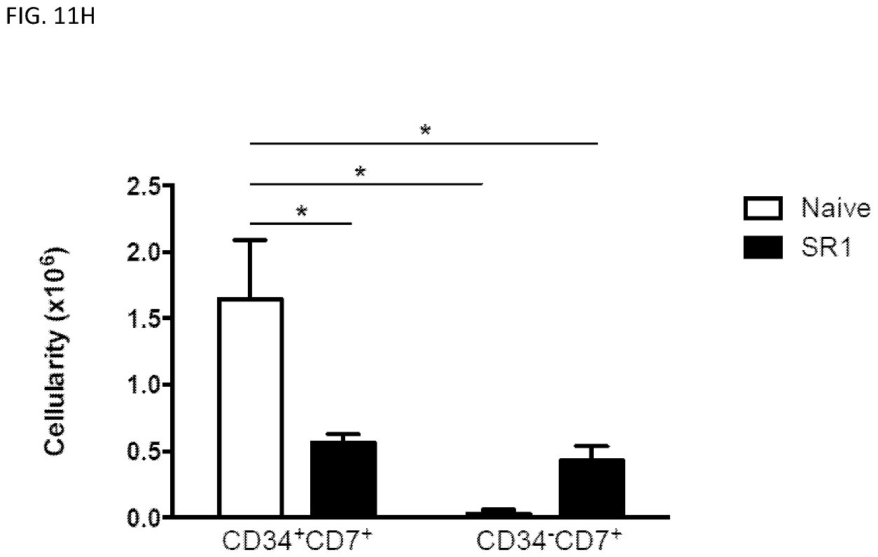

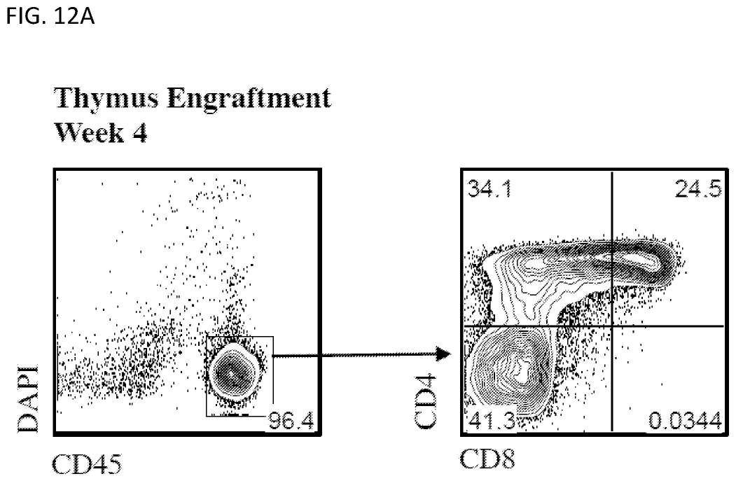

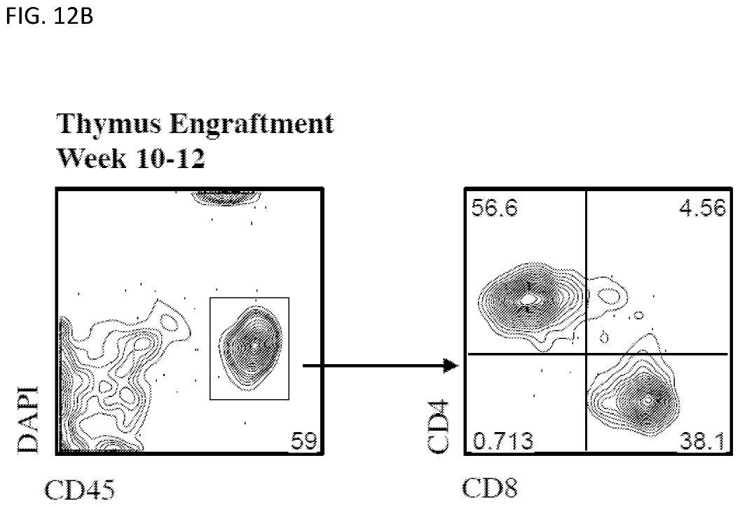

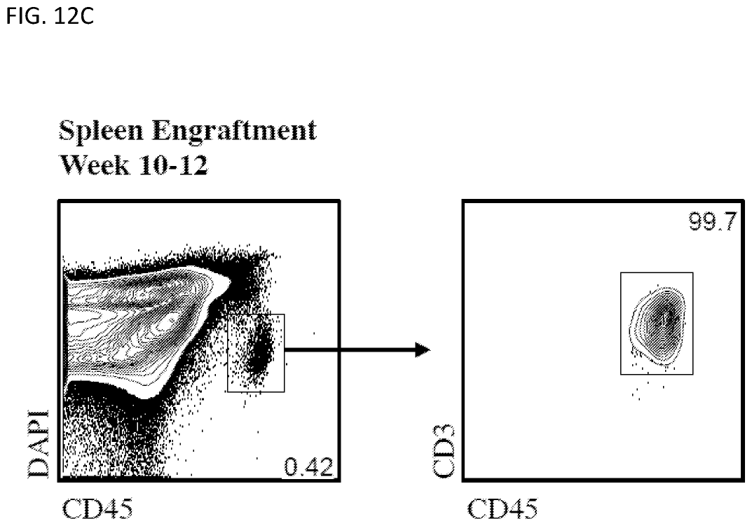

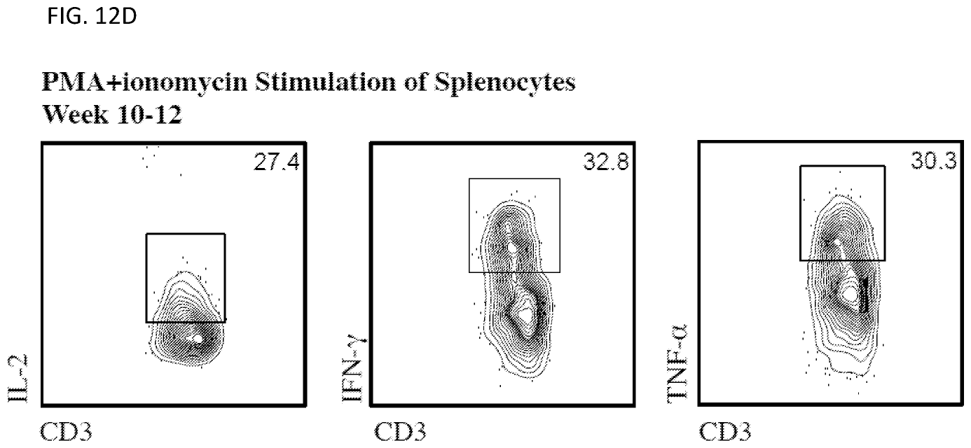

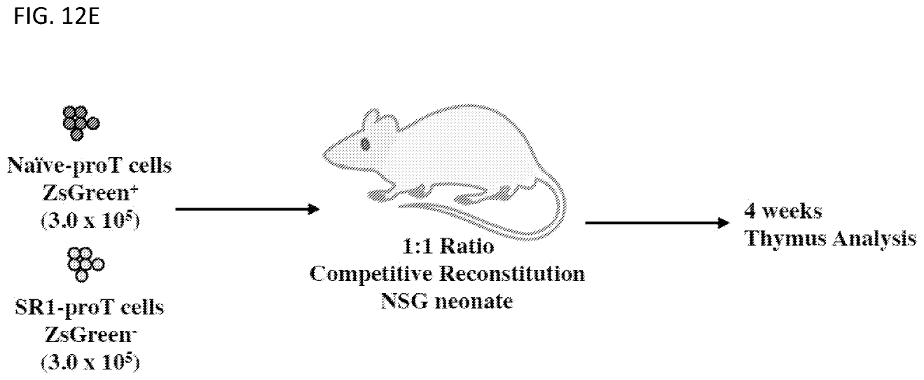

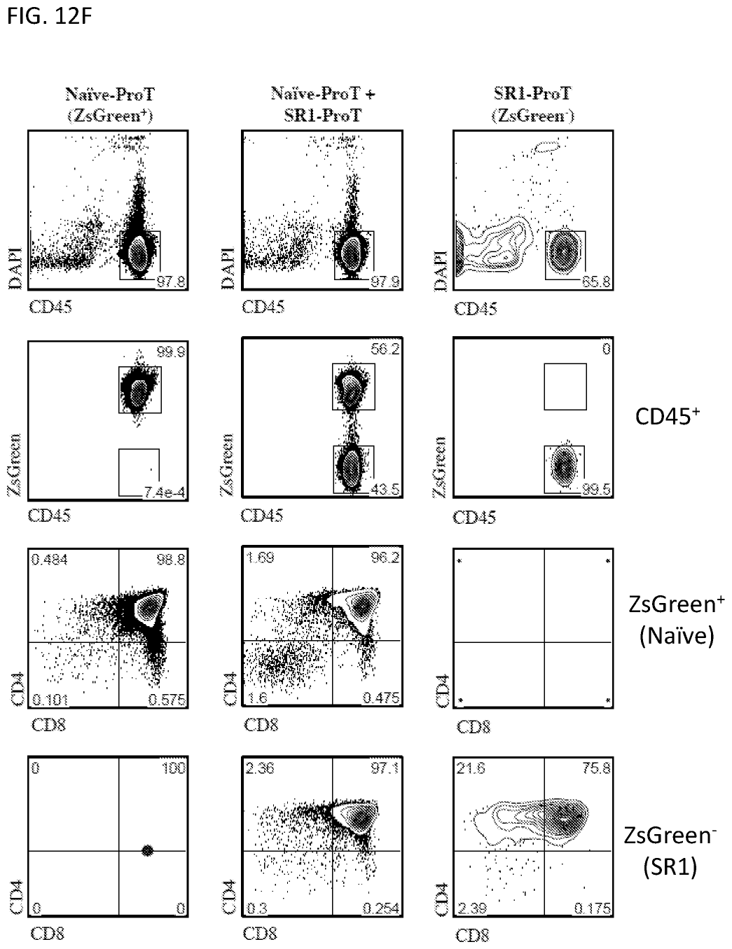

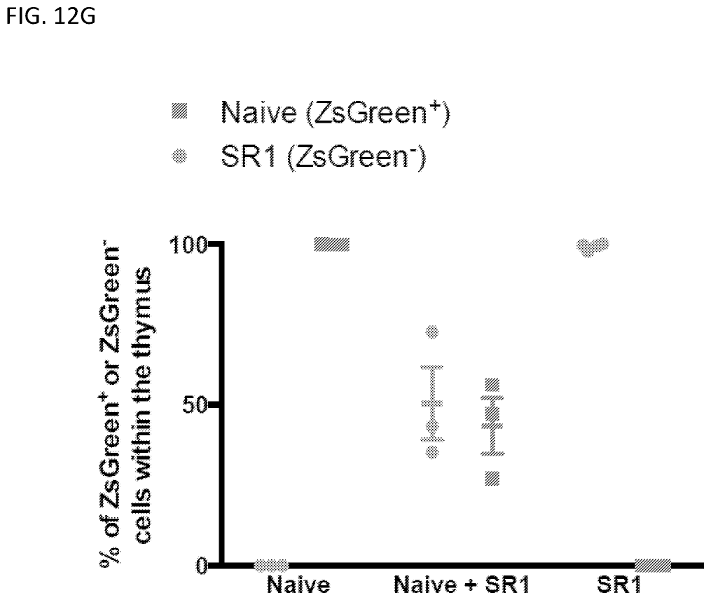

It has previously been reported that the only cells capable of engrafting the thymus are CD34⁺CD7⁺ cells (see, e.g., Awong et al., Blood 2009; 114(5):972-82), and that, to optimize the resulting number CD34⁺ cells available for engraftment, cells should be selected and/or purified for CD34⁺ cells prior to culture with a cell that expresses a Notch ligand. As shown, in Comparative Example 1 and FIG. 10, and Example 7 and FIG. 11G, although CD34⁺CD7⁺ cells generated from naïve UCB with OP9-DL1 cells can engraft in the thymus, CD34⁻CD7⁺ cells cannot. However, in some embodiments, this disclosure provides a method that preferably does not include a selecting for a CD34⁺ cell from an expanded population of cells prior to culturing the expanded population of cells with a cell that expresses a Notch ligand. Surprisingly, despite not being selected for CD34 expression after SR1 expansion, SR1-expanded cells generate logs-fold more ProT cells when cultured with a cell that expresses a Notch ligand than naïve umbilical cord blood cells selected for CD34 expression. Also unexpectedly, and as shown in FIG. 9, FIG. 12, and as further described in Example 7, CD34⁻CD7⁺ SR1-expanded cells generated from UCB with OP9-DL1 cells can engraft in the thymus.

After expansion, the expanded population of cells is cultured with a cell that expresses a Notch ligand. In some embodiments, a cell that expresses a Notch ligand includes an OP9 cell. In some embodiments, the Notch ligand includes delta-like 1 (DLL1 or DL1) or delta-like 4 (DLL4 or DL4). In some embodiments, a cell that expresses a Notch ligand includes OP9-DL1 or OP9-DL4. Such a co-culture may be performed using any suitable method including, for example, co-culture on a cell culture plate or in a cell culture flask.

The method further includes generation of a progenitor T cell from the culture of the expanded population of cells with the cell that expresses a Notch ligand.

In some embodiments, a progenitor T cell resulting from the culture of an expanded population of cells with a cell that expresses a Notch ligand expresses a diminished level of CD34 expression compared to a cell resulting from the culture of a non-expanded population of cells from the same source with a cell that expresses a Notch ligand. In some embodiments, CD34 expression is diminished by at least 50%, at least 60%, at least 70%, at least 75%, at least 80%, or at least 90%. In some embodiments, a population of cells including the progenitor T cell includes at least 80% cells, at least 90% cells, at least 95% cells, at least 97% cells, at least 98% cells, or at least 99% cells that are CD34⁻ cells. In some embodiments, a population of cells including a progenitor T cell resulting from the culture of an expanded population of cells with a cell that expresses a Notch ligand includes at least 80% cells, at least 90% cells, at least 95% cells, at least 97% cells, at least 98% cells, at least 99% cells that are CD34⁻CD7⁺ cells.

In some embodiments, this disclosure provides a method that does not include selection of CD34⁺ cells. Such selection (or lack thereof) could occur before culturing the expanded population of cells with a cell that expresses a Notch ligand or after culturing the expanded population of cells with a cell that expresses a Notch ligand.

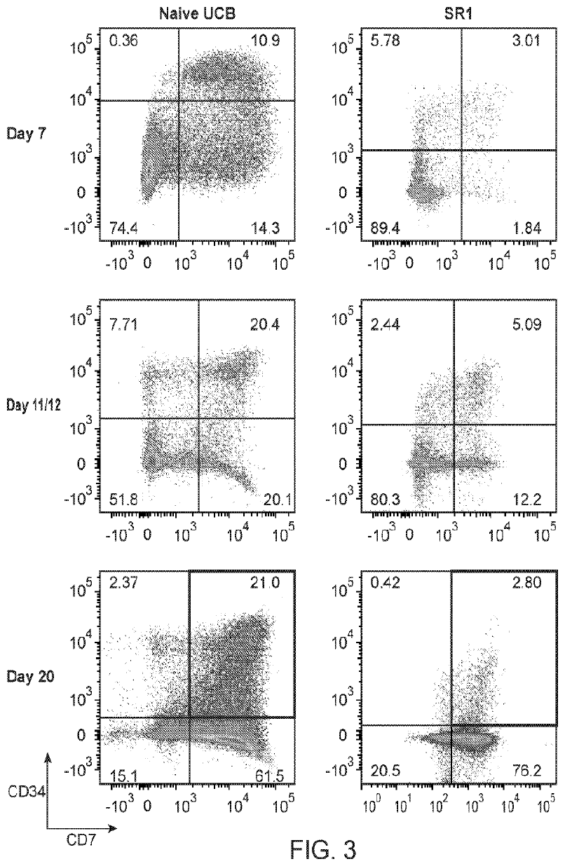

It has previously been reported that the only cells that have the ability to engraft the thymus are Cd34⁺CD7⁺, and that, to optimize the resulting number CD34⁺ cells available for engraftment, cells should be selected and/or purified for expression of CD34 and CD7 after culture with a cell that expresses a Notch ligand and/or prior to engraftment. However, as shown, for example, in FIG. 3-FIG. 6, despite not being selected for CD34⁺ cells and despite expressing a lower frequency of CD34⁺ cells than cells resulting from the culture of a non-expanded population of cells with a cell that expresses a Notch ligand, the expanded population of cells, after culturing with a cell that expresses a Notch ligand, surprisingly displays engraftment equivalent to that of a population of cells resulting from the culture of a non-expanded population of cells with a cell that expresses a Notch ligand.

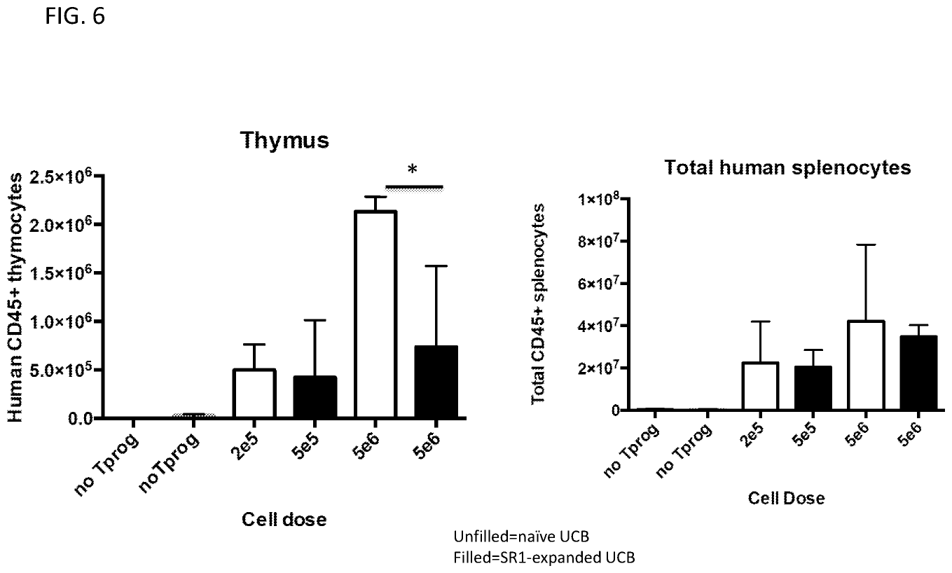

As shown in FIG. 6, despite the lack of expression of CD34, progenitor T cells generated from culturing SR1-expanded cells with a cell that expresses a Notch ligand demonstrate both thymic and peripheral T cell engraftment at levels consistent with the engraftment of CD34⁺ progenitor T cells generated from culturing non-expanded (i.e., naïve umbilical cord blood) with a cell that expresses a Notch ligand.

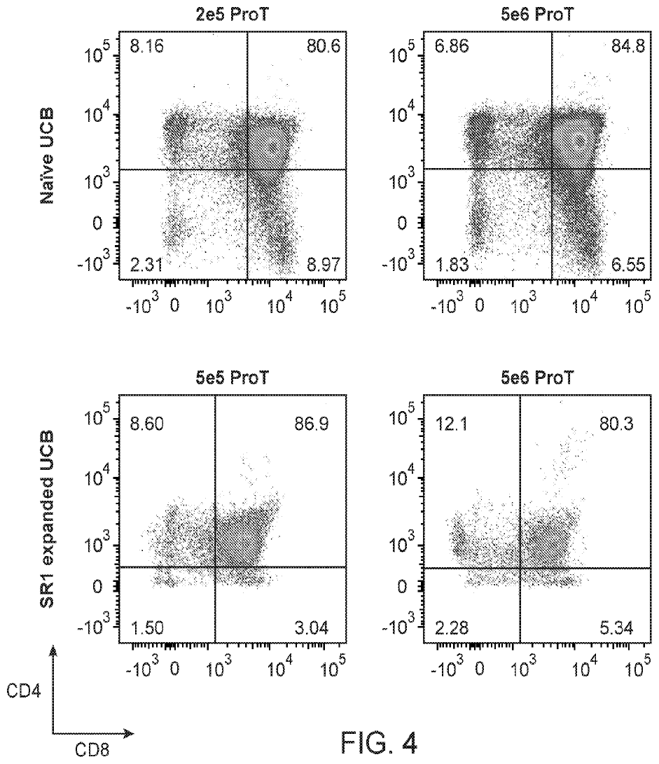

For example, mice that received 2×10⁵ total cells generated from naïve umbilical cord blood (UCB), received 4×10⁴ CD34⁺CD7⁺ cells, and mice that received 5×10⁵ total cells generated from SR1-expanded umbilical cord blood, received only 1×10⁴ CD34⁺CD7⁺ cells. U.S. Pat. No. 8,772,028 reports that CD34⁺CD7⁺cells are necessary for thymic engraftment. Thus, one would have expected 4-fold better engraftment of cells derived from naïve umbilical cord blood. Surprisingly, and as shown, for example, in FIG. 4 and FIG. 6, cells from naïve umbilical cord blood and cells generated from SR1-expanded umbilical cord blood engraft equally well in most circumstances. Although when 5×10⁶ total cells were administered, a 7.5-fold advantage of CD34⁺CD7⁺ cells was seen for cells derived from naïve UCB compared to cells derived from SR1-expanded UCB, no significant differences in thymic recovery were seen, and peripheral (spleen) T cell recovery was not adversely affected. Surprisingly, this shows that CD7⁺cells can engraft the thymus and further purification for CD34 expression is not required.

Moreover, as shown in FIG. 9, progenitor T cells generated from culturing SR1-expanded cells with a cell that expresses a Notch ligand demonstrate thymic engraftment despite not expressing CD34. In contrast, as shown in FIG. 10, progenitor T cells generated from non-SR1-expanded cells co-cultured with a cell that expresses a Notch ligand do not demonstrate thymic engraftment if they do not express CD34.

In some embodiments, the method including producing a progenitor T cell. further includes generating a derivative of the progenitor T cell. The derivative of the progenitor T cell may be generated in vivo or in vitro. In some embodiments, the derivative of the progenitor T cell includes a mature T cell. In some embodiments, the derivative of the progenitor T cell includes a cell that expresses CD3. In some embodiments, the derivative of the progenitor T cell includes a cell that expresses a T cell receptor. In some embodiments, the derivative of the progenitor T cell includes a cell that expresses one or more of CD3, an αβ T cell receptor, and a γδ T cell receptor. In some embodiments, the derivative of the progenitor T cell may be genetically modified.

In another aspect, this disclosure describes a progenitor T cell including, for example, a CD7⁺ progenitor T cell. In some embodiments, the progenitor T cell is preferably produced by a method disclosed herein. In some embodiments, the progenitor T cell is a CD7⁺CD34⁻ progenitor T cell. In some embodiments, the progenitor T cell is capable of engrafting, for example, in the thymus or the spleen or both. In some embodiments, the progenitor T cell includes an aryl hydrocarbon receptor antagonist-expanded progenitor T cell including, for example, an SR1-expanded progenitor T cell.

In a further aspect, this disclosure describes a derivative of the progenitor T cell.

This disclosure further describes a composition. The composition may include a progenitor T cell or a derivative of the progenitor T cell. For example, the composition could include a pharmaceutical composition including a progenitor T cell and/or a derivative of the progenitor T cell and a pharmaceutically acceptable carrier.

In some embodiments, a pharmaceutical composition may also include hematopoietic stem/progenitor cells (HSPCs). In some embodiments, the HSPCs may be from the same umbilical cord blood as the progenitor T cell and/or a derivative of the progenitor T cell.

In some embodiments, a pharmaceutical composition may include a solution including a progenitor T cell and/or a derivative of the progenitor T cell in association with one or more pharmaceutically acceptable vehicles or diluents and contained in a buffered solution that has a suitable pH and is iso-osmotic with the physiological fluids.

A pharmaceutical composition may include, without limitation, a lyophilized powder or an aqueous or non-aqueous sterile injectable solution or suspension, which may further contain an antioxidant, buffer, bacteriostat, and/or solute that render the composition substantially compatible with a tissue or the blood of an intended recipient. Other components that may be present in such compositions include water, a surfactant (including, for example, TWEEN), an alcohol, a polyol, a glycerin, and/or a vegetable oil, for example. An extemporaneous injection solution or suspension may be prepared from a sterile powder, a granule, a tablet, or a concentrated solution or suspension. The composition may be supplied, for example, as a lyophilized powder which is reconstituted with sterile water or saline prior to administration to the patient.

In some embodiments, such compositions should contain a therapeutically effective number of progenitor T cells and/or derivatives of the progenitor T cell, together with a suitable amount of a pharmaceutically acceptable carrier so as to provide a form for direct administration to a patient.

Suitable pharmaceutically acceptable carriers are described, for example, in Remington's Pharmaceutical Sciences. The pharmaceutically acceptable carrier may include, for example, an excipient, a diluent, a solvent, an accessory ingredient, a stabilizer, a protein carrier, or a biological compound. In some embodiments, suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, a saline solution, a glycerol solution, ethanol, N-(1(2,3-dioleyloxy)propyl) N,N,N-trimethylammonium chloride (DOTMA), diolesyl-phosphotidyl-ethanolamine (DOPE), and a liposome. Non-limiting examples of a protein carrier includes keyhole limpet hemocyanin (KLH), bovine serum albumin (BSA), ovalbumin, or the like. Non-limiting examples of a biological compound which may serve as a carrier include a glycosaminoglycan, a proteoglycan, and albumin. The carrier may be a synthetic compound, such as dimethyl sulfoxide or a synthetic polymer, such as a polyalkyleneglycol. Ovalbumin, human serum albumin, other proteins, polyethylene glycol, or the like may be employed as the carrier. In some embodiments, the pharmaceutically acceptable carrier includes at least one compound that is not naturally occurring or a product of nature.

In a further aspect, this disclosure describes a method of using a progenitor T cell and/or a derivative of the progenitor T cell. Such a method may include, for example a method of administering a cell. In some embodiments, a method of administering the cell may include administering a pharmaceutical composition. In some embodiments, the pharmaceutical composition includes a progenitor T cell and/or a derivative of the progenitor T cell and a pharmaceutically acceptable carrier. In some embodiments, the progenitor T cell and/or a derivative of the progenitor T cell is preferably administered in a therapeutically effective amount. In some embodiments, the progenitor T cell and/or a derivative of the progenitor T cell may be allogenic. When the cell is allogenic, the donor of the stem cells or progenitor cells may be selected on the basis of HLA match with the receiving patient. In some embodiments, the progenitor T cell and/or a derivative of the progenitor T cell may be autologous, for example, derived from the patient's own stem cells or progenitor cells.

In some embodiments, the progenitor T cell and/or a derivative of the progenitor T cell may be administered in combination with another therapy. For example, in some embodiments, it may be preferable to administer the progenitor T cell with umbilical cord blood (UCB) cells. The UCB cells may include, for example, CD34⁺ cells enriched from UCB, hematopoietic stem cells (HSCs), and/or hematopoietic stem/progenitor cells (HSPCs). In some embodiments, the progenitor T cell and/or a derivative of the progenitor T cell may be derived from the same umbilical cord as the co-administered UCB cells. In some embodiments, it may be preferable to administer the progenitor T cell and/or a derivative of the progenitor T cell with hematopoietic stem/progenitor cells (HSPCs).

In some embodiments, the UCB cells may be aryl hydrocarbon receptor antagonist-expanded including, for example, SRI-expanded. For example, HSCs or HSPCs could be aryl hydrocarbon receptor antagonist-expanded. In some embodiments, the progenitor T cell and/or a derivative of the progenitor T cell may be derived from the same umbilical cord as the co-administered UCB cells. For example, a progenitor T cell and/or a derivative of the progenitor T cell may be co-administered with aryl hydrocarbon receptor antagonist-expanded HSCs or HSPCs derived from the same UCB as the progenitor T cell and/or the derivative of the progenitor T cell.

T-cell lymphopenia is a critical risk factor for relapse post-hematopoietic stem cell transplantation. Managing T-cell reconstitution using an allogeneically-compatible transplant strategy remains important. Hematopoietic stem/progenitor cells (HSPCs) expansion using, for example, SR1 allows for increased HSPCs and proT-cells generation from the same unit. The results of Example 7 suggest that proT-cell infusion has the potential to confer rapid T-cell based immunity post-hematopoietic stem cell transplantation. Without wishing to be bound by theory, it is believed that proT-cells have intrinsic thymus-homing capacity, allowing them to restore short-term

T-cell-mediated immunity and reorganize thymic microenvironment, promoting lifelong HSPC-derived T-cell production. Notably, SR1-CD7⁺-cells co-injected with SR1-HSPC increased thymus engraftment more than 5 times compared to SR1-HSPC alone.

As described in Example 7, SR1-HSPC generated predominantly CD34⁻CD7⁺ cells after 14-day OP9-DL1 co-culture. Since both SR1-HSPC-derived CD7-expressing CD34⁺ and CD34⁻ subsets can engraft the thymus in vivo, a larger proT-cell product (compared to generation using naïve-HSPC can be generated for patients.

In some embodiments, the UCB cells may be selected on the basis of HLA match with the receiving patient and/or with the progenitor T cell. For example, in some embodiments, on the basis of antigen level HLA typing for A and B and allele level typing for DRB1, the progenitor T cell may be matched with the umbilical cord, the UCB cells, the HCSs, the HSPCs and/or the patient at at least 3 of 6 loci, at least 4 of 6 loci, at least 5 of 6 loci, or 6 of 6 loci.

In some embodiments, the progenitor T cell and/or a derivative of the progenitor T cell may be administered to a patient in need of a hematopoietic stem cell transplant or a patient having a condition requiring an increase in the number of T cells. Such patients may include, for example, a patient having undergone an organ transplant; a patient exhibiting a lymphopenia; a patient having a cancer such as multiple myeloma, leukemia, sarcoma, lymphoma, etc.; a patient having an autoimmune disease such as multiple sclerosis; a patient having an immunodeficiency; a patient having a skeletal dysplasia; a patient having a thalassemia; a patient having a hemoglobinopathy, a patient exhibiting anemia including, for example, sickle cell anemia, aplastic anemia, Faconi anemia; a patient exhibiting a bone marrow failure syndrome; and a patient exhibiting a genetic disorder including but not limited to Hurler syndrome, adrenal leukodystrophy, or epidermolysis bullosa.

In some embodiments, for example, in mice, at least 0.1×10⁶ progenitor T cells per kilogram (cells/kg), at least 0.3×10⁶ progenitor T cells/kg, at least 1×10⁶ progenitor T cells/kg, at least 4×10⁶ progenitor T cells/kg, or at least 5×10⁶ progenitor T cells/kg may be administered. In some embodiments, for example in humans, at least 0.1×10⁶ progenitor T cells/kg, at least 0.3×10⁶ progenitor T cells/kg, at least 1×10⁶ progenitor T cells/kg, or at least 4×10⁶ progenitor T cells may be administered. In some embodiments, in mice, successful engraftment is considered at least 0.5%, at least 0.75%, at least 1%, or at least 1.25% human CD45⁺ cells in the spleen or thymus or both. In some embodiments, in mice, successful engraftment is preferably considered at least 1% human CD45⁺cells. In some embodiments, successful engraftment is considered at least 0.5%, at least 0.75%, at least 1%, or at least 1.25% engrafted CD45⁺cells. Similar ranges may also apply for administration of a derivative of the progenitor T cell.

In some embodiments, producing the progenitor T cell using a method that includes expanding the cells prior to co-culture allows the co-administration of umbilical cord blood stem cells and progenitor T cells derived from the same umbilical cord. Such co-administration with progenitor T cells derived from naïve UCB (i.e., a non-expanded population of cells) is not practical because even using an entire UCB cord blood unit, the number of progenitor T cells obtained may not be therapeutically relevant. In contrast, by using progenitor T cells derived from expanded UCB (i.e., an expanded population of cells), a therapeutically relevant number of progenitor T cells may be obtained and part of the UCB cells may be reserved for administration with the progenitor T cells and/or a derivative of the progenitor T cell.

A composition of this disclosure may be administered for example, by parenteral, intravenous, subcutaneous, intramuscular, intracranial, intraorbital, ophthalmic, intraventricular, intracapsular, intraspinal, intracisternal, intraperitoneal, intranasal, aerosol, or oral administration. In some embodiments, a compositions of may be administered by injection into the liver. For parenteral administration, solutions that include a progenitor T cell may be prepared in water suitably mixed with a surfactant such as hydroxypropylcellulose. Dispersions may also be prepared in glycerol, liquid polyethylene glycols, DMSO, and mixtures thereof with or without alcohol, and in oils. Under ordinary conditions of storage and use, these preparations may contain a preservative to prevent the growth of microorganisms. A person skilled in the art would know how to prepare suitable formulations.

Preferably the progenitor T cell and/or a derivative of the progenitor T cell is present in an amount effective for treating a disease state in a mammal in need thereof In one embodiment, the progenitor T cell is present in an amount effective to enhance hematopoietic progenitor cell engraftment in a mammal in need thereof. Optionally, the composition further comprises a tissue for transplantation. In one embodiment, the tissue comprises a thymus. In some embodiments, the tissue comprises an organ.

This disclosure further describes a method that includes administering the cells described herein to a subject. As used herein, the term “subject” represents an organism, including, for example, a mammal. A mammal includes, but is not limited to, a human, a non-human primate, and other non-human vertebrates. A subject may be an “individual,” a “patient,” or a “host.” Non-human vertebrates include livestock animals (such as, but not limited to, a cow, a horse, a goat, and a pig), a domestic pet or companion animal, such as, but not limited to, a dog or a cat, and laboratory animals. Non-human subjects also include non-human primates as well as rodents, such as, but not limited to, a rat or a mouse. Non-human subjects also include, without limitation, poultry, horses, cows, pigs, goats, dogs, cats, guinea pigs, hamsters, mink, and rabbits.

In some embodiments, administering the progenitor T cell and/or a derivative of the progenitor T cell described herein to a subject may include treating a subject having a condition requiring an increase in the number of T cells by administering an effective amount of a progenitor T cell. Such conditions may include, for example, a lymphopenia; a cancer including, for example, multiple myeloma, leukemia, sarcoma, lymphoma, etc.; an autoimmune disease such as multiple sclerosis; an immunodeficiency; a skeletal dysplasia; a thalassemia; a hemoglobinopathy; an anemia including, for example, sickle cell anemia, aplastic anemia, Faconi anemia; a bone marrow failure syndrome; and a genetic disorder including but not limited to Hurler syndrome, adrenal leukodystrophy, or epidermolysis bullosa.

In some embodiments, the progenitor T cell and/or a derivative of the progenitor T cell may be derived from the patient's own stem cells or progenitor cells. In some embodiments, the progenitor T cell and/or a derivative of the progenitor T cell may be derived from a source other than the patient. When the progenitor T cell and/or a derivative of the progenitor T cell is derived from a source other than the patient, the source may be selected based on HLA match between the source and the patient. For example, in some embodiments, HLA match will include determining the number of loci exhibiting a match for antigen level HLA typing for A and B and/or allele level typing for DRB1. In some embodiments, the patient and the source may exhibit an HLA match at least 3 of 6 loci, at least 4 of 6 loci, at least 5 of 6 loci, or 6 of 6 loci.

As used herein, the phrase “effective amount” or “therapeutically effective amount” means an amount effective, at dosages and for periods of time necessary to achieve the desired result. Effective amounts may vary according to factors such as the disease state, age, sex, and/or weight of the subject. The amount of a given cell preparation that will correspond to such an amount will vary depending upon various factors. Such as the pharmaceutical formulation, the route of administration, the type of disease or disorder, the identity of the subject or host being treated, and the like, but can nevertheless be routinely determined by one skilled in the art. An “effective amount” will preferably be an amount effective for the progenitor T cells and/or a derivative of the progenitor T cell to engraft the subject being treated.

The term “treating” or “treatment” as used herein and as is well understood in the art, means an approach for obtaining beneficial or desired results, including clinical results. Beneficial or desired clinical results may include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” may also mean prolonging survival as compared to expected survival if not receiving treatment. “Treating” and “treatment” as used herein also include prophylactic treatment.

A “condition requiring an increase in the number of T cells” includes any condition wherein T cell levels are reduced as compared to a healthy animal or human. Such conditions may include, for example, anemia, immunodeficiency, autoimmune disease, lymphopenia, cancer, a genetic disorder, an infectious disease, and autoimmunity.

The present invention is illustrated by the following examples. It is to be understood that the particular examples, materials, amounts, and procedures are to be interpreted broadly in accordance with the scope and spirit of the invention as set forth herein.

### EXAMPLES

### Materials

OP9-DL1 cells: OP9 cells (Riken BioResource Center, Tsukuba, Japan; described on the world wide web at www2.brc.riken.jp/lab/cell/detail.cgi?cell_no=RCB1124& type=1) retrovirally transduced to express the gene Delta-like 1 (DLL-1 or DL-1).

α-Modified Eagle's Medium (αMEM) (GIBCO 12561-056, ThermoFisher Scientific, Waltham, Mass.). Stored at 4° C.

Fetal bovine serum (FBS).

Heat-inactivated Fetal bovine serum (hiFBS). FBA heated at 56° C. for 30 min. Stored at 4° C.

Penicillin/streptomycin: 100× or 10,000 U/mL penicillin and 10,000 U/mL streptomycin (HYCLONE SV30010). Used at 1×. Stored at 4° C. once opened.

Phosphate-buffered saline (PBS) 1× without Ca²⁺/Mg²⁺ (GIBCO 14190-144).

Trypsin 2.5% (GIBCO 15090). Diluted with PBS to 0.25% solution. Stored at 4° C.

OP9 media: αMEM supplemented with 20% hiFBS and 1×penicillin/streptomycin.

FALCON 40 μm cell strainers (Product No. 352340).

70 millimeter (mm) nylon mesh filters (Catalog No. N70R, BioDesign Inc. of New York, Carmel, N.Y.).

Human IL-7 (Catalog No. 200-07, PeproTech, Rocky Hill, N,J.). Reconstituted at 5 mg/mL (1,000×) in OP9 media. Aliquoted and stored at −80° C.

Human FLT-3L (Catalog No. 308-FK, R&D Systems, Inc., Minneapolis, Minn.). Reconstituted at 5 mg/mL (1,000×) in OP9 media. Aliquoted and stored at −80° C.

Human SCF (Catalog No. 300-07, PeproTech, Rocky Hill, N.J.) Reconstitute at 30 mg/mL (1,000×) in OP9 media. Aliquoted and stored −80° C.

Thrombopoietin (TPO) (Catalog No. 300-18, PeproTech, Rocky Hill, N.J.).

Freezing media: 90% hiFBS, 10% dimethyl sulfoxide (DMSO). Sterile filtered through a 0.22μ filter.

HYCLONE Hank's Balanced Salt Solution (HBSS) 1×without phenol red, Ca²⁺ or Mg²⁺ (Catalog No. SH30268.01, GE Healthcare LifeSciences, Logan, Utah).

Sorting buffer: HBSS, 1% Bovine Serum Albumin (BSA) Fraction V (OMNIPUR 2890).

Fluorescent-labeled mAbs to human CD7 (clone M-T701), CD34 (clone 581), and CD38 (clone HIT2) (BD Biosciences, San Jose, Calif.).

Tissue culture ware (10 cm dishes, 6-well plates, cryovials), tissue culture treated (suggested: SARSTEDT or FALCON).

SR1 at 750 nanomolar (nM) (Sigma-Aldrich, St. Louis, Mo.).

### Example 1. Pre-Treatment/Expansion of Umbilical Cord Blood (UCB) with SR1

Frozen UCB units were thawed using standard methods (Rubinstein et al., Proc Natl Acad Sci USA. 1995; 92:10119-10122). The UCB unit was enriched for CD34⁺-cells using the CliniMACS Cell Selection Device (Miltenyi Biotec, Gladbach, Germany) following manufacturer's instructions, and the resulting CD34-enriched cell population was placed in expansion media at a concentration of 5×10³ cells per milliliter (cells/mL). The expansion culture media included SCF, FLT-3L, TPO, IL-6 (each at 50 ng/ml) and SR1 (750 nM). Cells were cultured in expansion culture media without the addition of antibiotics for 15 days; cytokines were replenished and cells were resuspended at 5×10³ cells/mL at day 7.

At day 15, the cells were harvested and co-cultured with OP9-DL1 cells, as described in Example 4.

### Example 2. Culturing of OP9-DL1 Cells

All incubations were performed in a standard, humidified, cell culture incubator at 37° C. in 5% CO₂. In addition, cells are centrifuged at 450×g for 5 minutes at room temperature, unless otherwise indicated.

1. A vial of frozen OP9-DL1 cells was thawed in a 37° C. water bath using a gentle swirling motion and then transferred slowly by adding drop-wise using a 1 mL pipette into a 15 mL conical tube containing OP9 media.
2. The cells were centrifuged to obtain a pellet then suspended in 9-10 mL of fresh OP9 media before being seeded in a 10 cm dish.
3. The medium was changed the following day. Cells were passed when the cultures were 80% to 90% confluent. Appropriate confluence was generally maintained by splitting cells 1:4 every 2 days.
4. To passage OP9-DL1 cells from a 10 cm plate, medium was removed and 5 mL PBS was added to wash off the remaining medium. PBS was removed and replaced with 5 mL 0.25% trypsin and incubated for 5 minutes at 37° C.
5. Following trypsinization, the cells were vigorously pipetted to remove them from the surface of the plate and transferred to a 15 mL conical tube containing 5 mL of OP9 media. The plate was rinsed with 5 mL of PBS and the PBS was added to the contents of the first collection. The cells were centrifuged, suspended in OP9 media, and divided among 10 centimeter (cm) and/or 6-well plates. Each plate was gently rocked back and forth to ensure even cell distribution.

### Example 3. Irradiating OP9 Cells

Harvested OP9 cells were irradiated at 10000 cGy following trypsinization but prior to co-culture.

### Example 4. Co-Culture of SR1-Expanded UCBs with OP9-DL1 Cells

1. SR1-expanded cells were suspended in 3 mL of OP9 media then seeded into a plate/flask containing irradiated OP9-DL1 cells at 80% confluency. The human cytokines FLT-3L, IL-7, and SCF were added from a 1,000×stock solution (to 1×final concentration).
2. Additional human cytokines FLT-3L, IL-7, and SCF were added from a 1,000×stock solution (to 1×final concentration) every other day during cell culture.
3. At day 5 and every 3 days to 4 days thereafter, media containing cells was removed, and cells and media were passed through a 70 μm sterile nylon mesh or a 40 μm cell strainer into a 50 mL conical tube. Passage through the mesh or strainer removes OP9-DL1 cells but not cells derived from UCBs. PBS (5 mL) was added, and the coculture was disaggregated by vigorous pipetting (using a 5 mL pipette) and passed through the same cell strainer. An additional 5 mL of

PBS was added to obtain the remaining cells from the 6-well plate and then passed through the same cell strainer.

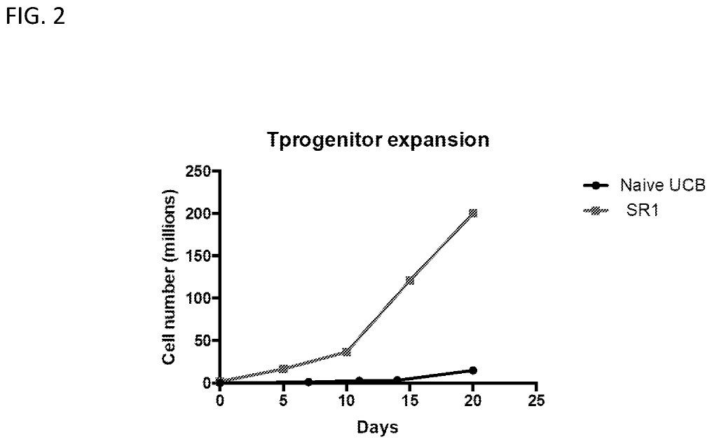

4. The cells that passed through the cell strainer were centrifuged at 515-585×g for 5 minutes, the supernatant was removed, and the cells were suspended in 1 mL of OP9 media. At this stage, the cells were counted using a hemocytometer (FIG. 2), assayed by flow cytometry (FIG. 3), or co-culture was continued. For continued co-culture, the cells were transferred into a new 6-well plate already containing OP9-DL1 cells at 80% confluency in 2 mL of OP9 media, and human cytokines FLT-3L, IL-7, and SCF were added from a 1,000×stock solution (to 1×final concentration).
5. Cells were harvested as described in steps 3 and 4 at day 14 or day 21 and phenotype was assessed (see FIG. 2, FIG. 3, and FIG. 7) or the cells were injected into a mouse, as described in Example 5.

### Example 5. Transfer to and Engraftment of T-progenitors in a Mouse

ProT cells generated as described in Example 4 (after 21 days of co-culture) were injected into the liver of 2 day to 5 day old neonatal (NOD/SCID/γcnull (NSG)) mice (3 mice per group) at different cell concentrations/mouse (e.g., 2×10⁵ cells, 5×10⁵ cells, or 5×10⁶ cells in 30 μL, volume). Optionally, 2×10⁴ CD34-enriched HSCs, isolated from a UCB unit using the CliniMACS Cell Selection Device (Miltenyi Biotec, Gladbach, Germany) following manufacturer's instruction, were injected simultaneously.

Twelve weeks later, mice were sacrificed, and engraftment of the in vitro-derived ProT cells into immunodeficient mice was assessed by flow cytometry analysis using phenotypic characterization of cells within the thymus and spleen of the recipient mouse. Useful markers for analysis include CD45, CD3/TCR, CD8, CD4, CD5, CD7, and CD1a (antibodies were acquired from eBioscience, San Diego, Calif.).

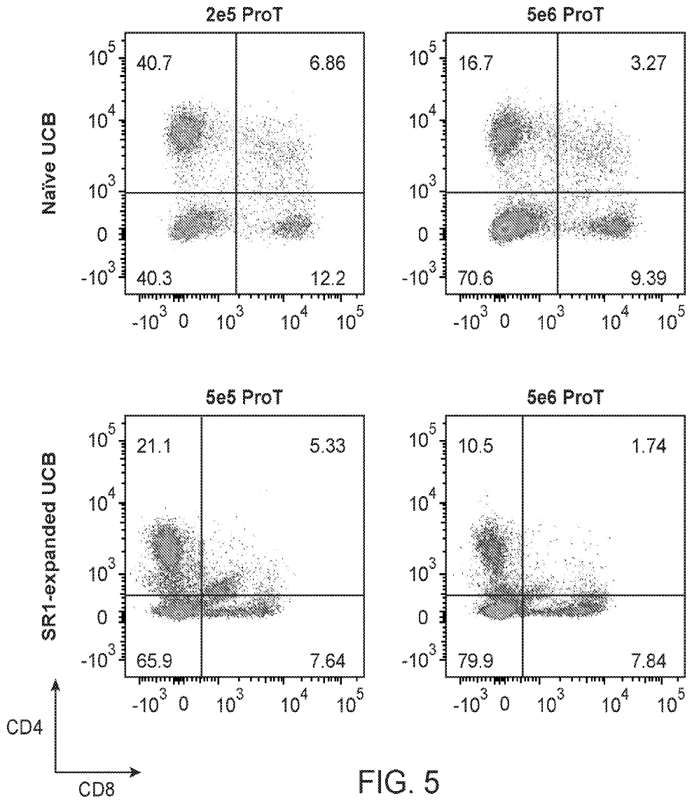

Results are shown in FIG. 4, FIG. 5, and FIG. 6.

### Example 6. Sorted CD34⁻CD7⁺ Tprogenitors from SR1-Expanded Cord Blood can Engraft in the Thymus

This Example shows CD34⁻CD7⁺ Tprogenitors from SR1-expanded cord blood result in human thymic and peripheral T cell engraftment.

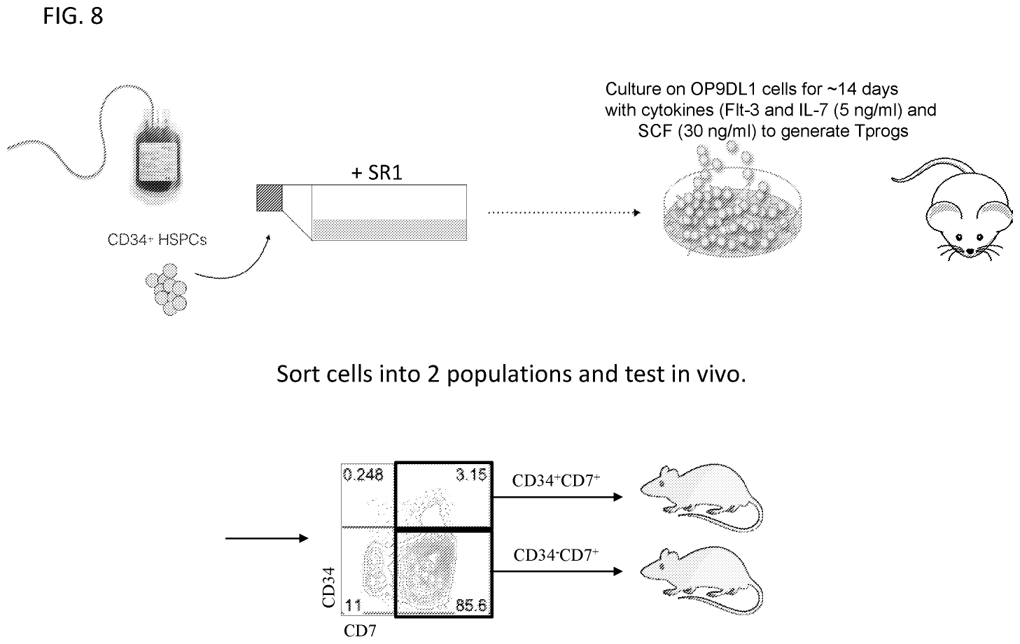

As shown schematically in FIG. 8, SR1-expanded CD34⁺ cells from a UCB unit were put into culture with OP9-DL1 cells for 14 days as described in Example 4. Cells were then sorted into two populations: CD34⁺CD7⁺ and CD34⁻CD7⁺. 1×10⁶ million cells of the resulting cell populations were injected into irradiated immunodeficient mice as described in Example 5. Mice were given rhIL-7 (0.5 μg) and anti-IL7 mAb, M25 (2.5 μg), in 20 μL of PBS (IL7+M25) three times weekly. Four weeks later the mice were sacrificed and the cells' ability to engraft the thymus was assessed.

Results are shown in FIG. 9. Surprisingly, CD34⁻CD7⁺ Tprogenitors from SR1-expanded cord blood can engraft in the thymus.

### Comparative Example 1. CD34⁻CD7⁺ Tprogenitors from Naïve UCB do not Engraft in the Thymus

Tprogenitors were generated from naïve UCB by co-culture with OP9-DL1 cells for 14 days as described in Example 4. Cells were sorted into two populations: CD34⁺CD7⁺ and CD34⁻CD7⁺. 1×10⁶ million cells of the resulting cell populations were injected into irradiated immunodeficient mice as described in Example 5. Mice were given rhIL-7 (0.5 μg) and anti-IL7 mAb, M25 (2.5 μg), in 20 μL of PBS (IL7+M25) three times weekly. Four weeks later the mice were sacrificed and the cells' ability to engraft the thymus was assessed.

Results are shown in FIG. 10. CD34⁻CD7⁺ Tprogenitors from naïve UCB (that is, non-SR1-expanded cord blood) do not engraft in the thymus.

### Example 7. Generation and Function of Progenitor T-cells from StemRegenin-1-Expanded CD34+ Human Hematopoietic Progenitor Cells

This Example describes methods that allow StemRegenin-1-(SR1)-expanded hematopoietic stem cells (HSCs) to give rise to large numbers of T-lineage cells in vitro and methods that allow CD34⁺CD7⁺as well as CD34⁻CD7⁺ from SR1-expanded HSCs to be effective thymus-reconstituting cells in vivo. More specifically, SR1-expanded umbilical cord blood (UCB) can induce greater than 250-fold expansion of CD34⁺ hematopoietic stem/progenitor cells (HSPCs) that generate large progenitor T (proT)-cell numbers in vitro. When compared to non-expanded naïve-proT-cells, SR1-proT-cells showed effective thymus-seeding and functional capabilities in vivo despite having an altered phenotype. In a competitive transfer approach, both nave and SR1-proT-cells showed comparable engrafting capacities. These findings support the use of SR1-expanded UCB grafts combined with proT-cell generation for decreasing T-cell immunodeficiency post-hematopoietic stem cell transplantation (HSCT).

### Methods

### Umbilical Cord Blood (UCB)

HSPC-containing purified fractions were purified from UCB (Awong et al. Blood. 2009; 114(5):972-982) under Research Ethics Board of Sunnybrook Health Sciences Centre approved guidelines.

### Mice

NOD.cg-Prkdc^{scid}IL2rg^{tm/Wjl}/Sz (NSG) mice purchased from Jackson Laboratory were housed and bred in a pathogen-free facility. Sunnybrook Health Sciences Centre Animal Care Committee approved the procedures.

### SR1-Expansion

HSC expansion media cultures (Boitano et al. Science. 2010; 329(5997):1345-1348) lasted 15 days prior to freezing.

### Tprogs

OP9-DL1 were gamma-irradiated (100 Gy) and seeded onto tissue culture flasks. SR1-UCB and naïve-UCB were seeded 2:1 with OP9-DL1 to generate proT-cells after 13-14 days (Schmitt et al. Immunity. 2002; 17(6):749-756).

### Engraftment

Sorted day 13-14 CD34⁺CD7⁺, CD34⁻CD7⁺, or bulk CD7⁺ cells from naïve- or SR1-UCB/OP9-DL1 cultures were injected (Awong et al. Blood. 2009; 114(5):972-982; Boyman et al. J Immunol. 2008; 180(11):7265-7275).

### Naïve/SR1-CD7+-Cell Coinjection

Naïve-UCB CD34⁺ cells were incubated in X-VIVO10 hematopoietic cell media (Lonza, Basel, Switzerland) containing TPO (10 ng/mL), Flt3L (100 ng/mL), SCF (100 ng/mL) and IL-3 (30 ng/mL). CD34⁺cells 1×10⁵) were added 24 hours later to Retronectin (20 μg/mL; Clontech Laboratories, Mountain View, Calif.)-coated plates with lentivirus (MOI, 50) for 24 hours. Sorted naïve-ZsGreen⁺ HSPC were placed on OP9-DL1 in parallel with SR1-HSPC. CD7⁺-proT-cells, 3×10⁵ of each subset, were coinjected into NSG neonates.

### Results & Discussion

### SRI Expanded Tprog T-Lymphopoietic Potential

It was previously reported that naïve-UCB can generate 4- to 5-fold Tprog expansion (Awong et al. Blood. 2009; 114(5):972-982). At least 2×10⁵ CD34⁺ HSPC are needed for HSCT; but a single UCB unit averages 2.5×10³ CD34⁺ HSPC (Delaney et al. Expert Rev Hematol. 2010; 3(3):273-283). This Example describes an investigation of whether adding SR1 (0.75 μM) to expand CD34⁺ HSPC Wagner et al. Cell Stem Cell. 2016; 18(1):144-155) could improve in vitro pro-T generation. CD34⁺ HSPC culture reproducibility (FIG. 11A) was characterized. SR1 significantly enhanced total nuclear cells by day 15 (Range: 252-746-fold) and long-term reconstituting CD34⁺CD38⁻ (Range: 157-558 fold) and CD34⁺CD90⁺ (Range 170-415 fold) cells, consistent with previous findings (Boitano et al. Science. 2010; 329(5997):1345-1348); Doulatov et al. Cell Stem Cell. 2012; 10(2):120-136).

To examine whether SR1-expanded HSPC could generate proT-cells in vitro, SR1-HSPC were co-cultured with OP9-DL1 (FIG. 11A). Day 14 co-cultures revealed that CD34⁺ HSPC undergoing early T-cell differentiation acquired CD7 and subsequently CD5 and CD1a (FIG. 11B), defining T-lineage commitment (Awong et al. Blood. 2009; 114(5):972-982; Spits Nat Rev Immunol. 2002; 2(10):760-772). Interestingly, CD34 was expressed in a higher proportion on day 14 naïve-HSPC than SR1-HSPC co-cultures with significantly lower % CD34⁺CD7⁺ cells (FIG. 11C). SR1-HSPC and naïve-HSPC co-cultures had similar % CD34⁻CD7⁺ cells, % CD7⁺CD5⁺ cells and % CD7⁺CD1a⁺ cells (FIG. 11C). Similar results were achieved with OP9-DL4 co-cultures (Besseyrias et al. J Exp Med. 2007; 204(2):331-343). From SR1-HSPC, an approximately 4-fold expansion of pro-Tcells was observed, similar to naïve-HSPC co-cultures (FIG. 11D). Combining SR1-expansion of HSPCs and use of OP9-DL1 co-cultures, a single starting HSPC yielded approximately 2×10³ cells, a 200-fold greater expansion of SR1-proT cells over naïve proT-cells (FIG. 11E).

### Unique Thymus-Homing ProT-Cell Subset

Thymus-homing cells, identified as CD34⁺CD7⁺, are present in UCB or fetal bone marrow (Haddad et al. Immunity. 2006; 24(2):217-230) and can be generated in vitro using the OP9-DL1 co-culture system (Awong et al. Blood. 2009;114(5):972-982; Awong et al. Blood. 2013; 122(26):4210-4219). Since CD34⁻CD7⁺ predominated over CD34⁺CD7⁺ cells in SR1-HSPC co-cultures, both populations were tested for thymus-reconstituting ability: sorted CD34⁻CD7⁺ or CD34⁺CD7⁺ cells from day 14 naïve- or SR1-HSPC/OP9-DL1 co-cultures were intra-hepatically injected into nonirradiated NSG neonatal mice and analyzed 4 weeks later (FIG. 11F). NSG mice receiving CD34⁺CD7⁺ cells from naïve- or SR1-HSPC displayed 95% human CD45⁺ cells in the thymus (FIG. 11G). While CD34⁻CD7⁺ cells from naïve-HSPC failed to engraft, clear detectable engraftment was seen from SR1-CD34⁻CD7⁺ cells, albeit 19-fold lower than their CD34⁺ counterparts, representing a novel functional capacity of CD34⁻CD7⁺ SR1-proT-cells, compared to CD34⁻CD7⁺ naïve-proT-cells. The majority of CD45⁺ cells in mice receiving either subset progressed along the T-lineage, with CD4⁺CD8⁺ double positive (DP) cells comprising the majority of human thymocytes in engrafted mice (FIG. 11G). Thymus cellularity from NSG mice receiving naïve-HSPC or SR1-HSPC in vitro-derived CD34⁺CD7⁺ cells or CD34⁻CD7⁺ cells is shown in FIG. 11H.

### SR1-CD7⁺-Cells Home and Mature In Vivo

Thymus-reconstituting capacity of CD34⁺CD7⁺ and CD34⁻CD7⁺ SR1-HSPC-derived cells prompted redefining proT-cells generated from SR1-HSPC simply as SR1-CD7⁺. Sorted SR1-CD⁺ cells from day 14 OP9-DL1 co-cultures supported high CD45⁺ thymic engraftment and differentiation, including a large DP number (FIG. 12A). Recirculating CD4⁺ and CD8⁺ single-positive T-lymphocytes were seen at 10-12 weeks within the thymus (FIG. 12B), along with circulating CD45⁺CD3⁺ splenic T-cells (FIG. 12C), indicating SR1-proT-cell peripheral reconstitution in NSG mice. To confirm functional maturation, splenic CD3⁺T-cells were stimulated with PMA+ionomycin in vitro. High levels of IL-2, IFN-γ and TNF-α, were observed after stimulation (FIG. 12D).

### Naïve-proT vs SR1-proT Competitive Reconstitution

To address SR1-CD7⁺- and naïve-CD7⁺-cell thymus-homing capacity, both cell types were competitively transferred into NSG mice, with cells traced by differences in ZsGreen expression. In vitro-generated ZsGreen⁺naïve-CD7⁺ and ZsGreen⁻SR1-CD7⁺ cells were injected in a 1:1 ratio (3×10⁵ each) into neonatal mice and analyzed 4 weeks later (FIG. 12E). Both naïve-CD7⁺-derived and SR1-CD7⁺-derived cells were present in the thymus of reconstituted mice, indicated by CD45⁺ZsGreen⁺ (56.2%) and CD45⁺ZsGreen⁻ (43.5%) cells, respectively (FIG. 12F), with all cells having progressed to the DP stage. Importantly, when the percentages of naïve- and SR1-proT-cells were analyzed in thymi across mice, SR1-proT-cells were consistently present at comparable frequencies to naïve proT-cells (FIG. 12G).

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern.

The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method comprising:
culturing stem cells or progenitor cells with a compound that promotes expansion of CD34⁺ cells to produce an expanded population of cells; and
culturing the expanded population of cells with a cell that expresses a Notch ligand to produce a CD7⁺ progenitor T cell.

2. The method of claim 1, wherein the compound that promotes expansion of CD34⁺ cells comprises an aryl hydrocarbon receptor antagonist, SR1, an SR1-derivative, a pyrimidoindole derivative, UM171, or UM729, or a combination thereof

3.-5. (canceled)

6. The method of claim 1, wherein the CD7⁺ progenitor T cell
expresses CD1a, or CD5, or both; or
does not express CD34; or
both.

7.-9. (canceled)

10. The method of claim 1, wherein the Notch ligand comprises DL1 or DL4 or both; or wherein the cell that expresses a Notch ligand comprises an OP9 cell; or wherein the cell that expresses a Notch ligand comprises an OP9-DL1 cell or an OP9-DL4 cell.

11.-12. (canceled)

13. The method of claim 1, wherein the method does not comprise selection of CD34⁺ cells.

14. The method of claim 1, wherein the stem cells comprise hematopoietic stem cells.

15. The method of claim 1, wherein the expanded population of cells comprises at least 90% CD34⁻ cells.

16. The method of claim 1, wherein the method further comprises
isolating the stem cells or progenitor cells from umbilical cord blood, peripheral blood, an induced pluripotent stem cell (iPSC), an embryonic stem cell, or bone marrow, or a combination thereof; or
administering the CD7⁺ progenitor T cell to a mammal, or
both.

17. (canceled)

18. The method of claim 16, wherein the method further comprises administering umbilical cord blood (UCB) cells CD34⁺ cells enriched from umbilical cord blood, or hematopoietic stem cells (HSCs), or a combination thereof to a mammal.

19.-20. (canceled)

21. The method of claim 18, wherein the method comprises expanding HSCs by treatment with aryl hydrocarbon receptor antagonist prior to administering the HSCs.

22. The method of claim 18, wherein the stem cells or progenitor cells are derived from umbilical cord blood and further wherein the umbilical cord blood cells, CD34⁺ cells enriched from umbilical cord blood, and/or the HSCs are derived from the same umbilical cord.

23. A CD7⁺ progenitor T cell produced by the method of claim 1.

24. A composition comprising the CD7⁺ progenitor T cell of claim 23, the composition further comprising umbilical cord blood (UCB) cells.

25. (canceled)

26. The method of claim 1, wherein the method further includes generating a derivative of the progenitor T cell, wherein the derivative of the progenitor T cell comprises a cell that expresses CD3 and a T cell receptor.

27.-29. (canceled)

30. A composition comprising a derivative of the CD7⁺ progenitor T cell produced by the method of claim 26, the composition further comprising umbilical cord blood (UCB) cells.

31. (canceled)

32. A method comprising administering the composition of claim 24 to a mammal, wherein the mammal has a condition requiring an increase in the number of T cells.

33. A method comprising administering the composition of claim 30 to a mammal, wherein the mammal has a condition requiring an increase in the number of T cells.

34. (canceled)

35. An isolated CD34⁻CD7⁺ progenitor T cell.

36. (canceled)

37. The progenitor T cell of claim 35, wherein the CD34⁻ CD7⁺ progenitor T cell comprises an aryl hydrocarbon receptor antagonist-expanded CD34⁻-CD7⁺ progenitor T cell or a pyrimidoindole derivative-expanded CD34⁻ CD7⁺ progenitor T cell or both.

38. The progenitor T cell of claim 37, wherein the CD34⁻CD7⁺ progenitor T cell comprises an SR1-expanded CD34⁻CD7⁺ progenitor T cell or a UM171-expanded CD34⁻CD7⁺ progenitor T cell.

39. (canceled)
